(19) United States
(12) Patent Application Publication
(10) Pub. No.: US 2021/0312156 A1
(43) Pub. Date: Oct. 7, 2021

(54) LIGHT DETECTING ELEMENT AND FINGERPRINT AUTHENTICATING DEVICE

(71) Applicant: SUMITOMO CHEMICAL COMPANY, LIMITED, Tokyo (JP)

(72) Inventors: Giovanni FERRARA, Tsukuba-shi (JP); Takahiro SEIKE, Tsukuba-shi (JP)

(73) Assignee: SUMITOMO CHEMICAL COMPANY, LIMITED, Tokyo (JP)

(21) Appl. No.: 17/264,238

(22) PCT Filed: Jul. 26, 2019
(86) PCT No.: PCT/JP2019/029403

§ 371 Date: Jan. 28, 2021

(30) Foreign Application Priority Data
Aug. 1, 2018 (JP) 2018-145299

(51) Int. Cl. G06K 9/00; H01L 51/44; H01L 51/00

(52) U.S. Cl. CPC G06K 9/0004; H01L 51/442; B82Y 20/00; H01L 51/0034; H01L 51/0007; H01L 51/0046

(57) ABSTRACT

Provided is a light detecting element having a low dark current. This light detecting element comprises: a first electrode and a second electrode; an active layer provided between the first electrode and the second electrode; and a hole transport layer provided between the second electrode and the active layer. The active layer contains an organic compound and has a thickness of 600 nm or more. The hole transport layer contains nanoparticles of metal oxide. The metal oxide includes one or more species selected from the group consisting of a molybdenum atom, a tungsten atom, and a nickel atom.

### TECHNICAL FIELD

The present invention relates to a light detecting element and a fingerprint authenticating device.

### BACKGROUND ART

The photoelectric conversion element has been attracting attention as a very useful device from the viewpoint of energy saving and reduction of carbon dioxide emissions, for example.

In order to improve the photoelectric conversion efficiency of the photoelectric conversion element, a technique of incorporating predetermined metal oxide nanoparticles in a buffer layer of the photoelectric conversion element is disclosed (Patent Document 1).

### PRIOR ART DOCUMENTS

### Patent Document

Patent Document 1: WO 2016/128133

### SUMMARY OF THE INVENTION

### Problems to be Solved by the Invention

A light detecting element, which is a type of photoelectric conversion element, is required to have a low dark current, that is, the current generated in a state where light is not irradiated, in order to improve the detection sensitivity.

On the other hand, in recent years, a light detecting element (organic light detecting element) using an organic material as a material of an active layer has been researched and developed. However, the organic light detecting element in the related art cannot sufficiently reduce the dark current.

Therefore, there is a demand for a light detecting element with a low dark current.

### Means for Solving the Problems

In order to solve the above problems, the present inventors have diligently studied. As a result, they have found that the above-described problems can be solved by a light detecting element in which an active layer has a predetermined thickness and a hole transport layer contains nanoparticles of metal oxide, and have completed the present invention. That is, the present invention provides the following.

[1] A light detecting element including a first electrode, and a second electrode,

an active layer provided between the first electrode and the second electrode, and

a hole transport layer provided between the second electrode and the active layer,

wherein the active layer contains an organic compound, and the thickness of the active layer is 600 nm or more, and

the hole transport layer contains nanoparticles of metal oxide.

[2] The light detecting element according to [1], wherein the metal oxide contains at least one selected from the group consisting of a molybdenum atom, a tungsten atom, and a nickel atom.

[3] The light detecting element according to [2], wherein the metal oxide is at least one selected from the group consisting of molybdenum oxide, tungsten oxide, and nickel oxide.

[4] The light detecting element according to any one of [1] to [3], wherein the active layer contains a conjugated polymer compound.

[5] The light detecting element according to any one of [1] to [4], wherein the active layer contains a fullerene derivative.

[6] A fingerprint authenticating device including the light detecting element according to any one of [1] to [5].

### Effect of the Invention

According to the present invention, it is possible to provide a light detecting element having a low dark current and a fingerprint authenticating device including the light detecting element.

### BRIEF DESCRIPTION OF THE DRAWINGS

FIG. 1 is a diagram schematically illustrating a light detecting element according to an embodiment of the present invention.

FIG. 2 is a diagram schematically illustrating a configuration example of an image detector.

FIG. 3 is a diagram schematically illustrating a configuration example of a fingerprint detector.

### MODE FOR CARRYING OUT THE INVENTION

Hereinafter, a light detecting element according to an embodiment of the present invention will be described with reference to the drawings. Note that the drawings merely outline the shapes, sizes and arrangements of components to the extent that the invention can be understood. The present invention is not limited to the following description, and each component can be appropriately modified without departing from the gist of the present invention. Further, in the configuration according to the embodiment of the present invention, it is not always produced or used in the arrangement illustrated in the drawings. In each of the drawings described below, the same components may be given the same sign and the description thereof may be omitted.

[1. Explanation of Common Terms]

The term “polymer compound” means a polymer having a molecular weight distribution and having a polystyrene-equivalent number average molecular weight of 1×10³ or more and 1×10⁸ or less. Constituent units contained in the polymer compound are 100 mol % in total.

The term “constituent unit” means a unit existing in one or more in a polymer compound.

A “hydrogen atom” may be a light hydrogen atom or a deuterium atom.

A “halogen atom” includes a fluorine atom, a chlorine atom, a bromine atom, and an iodine atom.

The meaning of “may have a substituent” includes a case where all the hydrogen atoms constituting the compound or group are unsubstituted, and a case where some or all of one or more hydrogen atoms are substituted by the substituent.

Unless otherwise specified, the “alkyl group” may be linear, branched, or cyclic. The number of carbon atoms of the linear alkyl group is usually 1 to 50, preferably 1 to 30, and more preferably 1 to 20 without including the number of carbon atoms of the substituent. The number of carbon atoms of a branched or cyclic alkyl group is usually 3 to 50, preferably 3 to 30, and more preferably 4 to 20 without including the number of carbon atoms of the substituent.

The alkyl group may have a substituent. Specific examples of the alkyl group include an alkyl group such as a methyl group, an ethyl group, an n-propyl group, an isopropyl group, an n-butyl group, an isobutyl group, a tert-butyl group, an n-pentyl group, an isoamyl group, a 2-ethylbutyl group, an n-hexyl group, a cyclohexyl group, an n-heptyl group, a cyclohexylmethyl group, a cyclohexylethyl group, an n-octyl group, a 2-ethylhexyl group, a 3-n-propylheptyl group, an adamantyl group, an n-decyl group, a 3,7-dimethyloctyl group, a 2-ethyloctyl group, a 2-n-hexyl-decyl group, an n-dodecyl group, a tetradecyl group, a hexadecyl group, an octadecyl group, and an eicosyl group; and an alkyl group having a substituent such as a trifluoromethyl group, a pentafluoroethyl group, a perfluorobutyl group, a perfluorohexyl group, a perfluorooctyl group, a 3-phenylpropyl group, a 3-(4-methylphenyl) propyl group, a 3-(3,5-di-n-hexylphenyl) propyl group, and a 6-ethyloxyhexyl group.

An “aryl group” means a remaining atomic group obtained by removing one hydrogen atom directly bonded to a carbon atom constituting a ring from an aromatic hydrocarbon which may have a substituent.

The aryl group may have a substituent. Specific examples of the aryl group include a phenyl group, a 1-naphthyl group, a 2-naphthyl group, a 1-anthrasenyl group, a 2-anthrasenyl group, a 9-anthrasenyl group, a 1-pyrenyl group, a 2-pyrenyl group, a 4-pyrenyl group, a 2-fluorenyl group, a 3-fluorenyl group, a 4-fluorenyl group, a 2-phenylphenyl group, a 3-phenylphenyl group, a 4-phenylphenyl group, and a group having a substituent such as an alkyl group, an alkoxy group, an aryl group, and a fluorine atom.

An “alkoxy group” may be linear, branched, or cyclic. The number of carbon atoms of the linear alkoxy group is usually 1 to 40 and preferably 1 to 10 without including the number of carbon atoms of the substituent. The number of carbon atoms of the branched or cyclic alkoxy group is usually 3 to 40 and preferably 4 to 10 without including the number of carbon atoms of the substituent.

The alkoxy group may have a substituent. Specific examples of the alkoxy group include a methoxy group, an ethoxy group, an n-propyloxy group, an isopropyloxy group, an n-butyloxy group, an isobutyloxy group, a tert-butyloxy group, an n-pentyloxy group, an n-hexyloxy group, a cyclohexyloxy group, an n-heptyloxy group, an n-octyloxy group, a 2-ethylhexyloxy group, an n-nonyloxy group, an n-decyloxy group, a 3,7-dimethyloctyloxy group, and a lauryloxy group.

The number of carbon atoms of the “aryloxy group” is usually 6 to 60 and preferably 6 to 48 without including the number of carbon atoms of the substituent.

The aryloxy group may have a substituent. Specific examples of the aryloxy group include a phenoxy group, a 1-naphthyloxy group, a 2-naphthyloxy group, a 1-anthrasenyloxy group, a 9-anthrasenyloxy group, a 1-pyrenyloxy group, and a group having a substituent such as an alkyl group, an alkoxy group, and a fluorine atom.

An “alkylthio group” may be linear, branched, or cyclic. The number of carbon atoms of the linear alkylthio group is usually 1 to 40 and preferably 1 to 10 without including the number of carbon atoms of the substituent. The number of carbon atoms of the branched and cyclic alkylthio groups is usually 3 to 40 and preferably 4 to 10 without including the number of carbon atoms of the substituent.

The alkylthio group may have a substituent. Specific examples of the alkylthio group include a methylthio group, an ethylthio group, a propylthio group, an isopropylthio group, a butylthio group, an isobutylthio group, a tert-butylthio group, a pentylthio group, a hexylthio group, a cyclohexylthio group, a heptylthio group, an octylthio group, a 2-ethylhexylthio group, a nonylthio group, a decylthio group, a 3,7-dimethyloctylthio group, a laurylthio group, and a trifluoromethylthio group.

The number of carbon atoms of an “arylthio group” is usually 6 to 60 and preferably 6 to 48 without including the number of carbon atoms of the substituent.

The arylthio group may have a substituent. Examples of the arylthio group include a phenylthio group and a C1 to C12 alkyloxyphenylthio group (here, “C1 to C12” indicates that the group to be described immediately after that has 1 to 12 carbon atoms. The same applies to the following.), a C1 to C12 alkylphenylthio group, a 1-naphthylthio group, a 2-naphthylthio group, and a pentafluorophenylthio group.

A “p-valent heterocyclic group” (p represents an integer of 1 or more) means a remaining atomic group excluding p hydrogen atoms among the hydrogen atoms directly bonded to a carbon atom or a hetero atom constituting a ring from a heterocyclic compound that may have a substituent. Among the p-valent heterocyclic groups, a “p-valent aromatic heterocyclic group” is preferable. The “p-valent aromatic heterocyclic group” means a remaining atomic group excluding p hydrogen atoms among the hydrogen atoms directly bonded to a carbon atom or a hetero atom constituting a ring from an aromatic heterocyclic compound that may have a substituent.

Examples of the substituent that the heterocyclic compound may have include a halogen atom, an alkyl group, an aryl group, an alkoxy group, an aryloxy group, an alkylthio group, an arylthio group, a monovalent heterocyclic group, a substituted amino group, an acyl group, an imine residue, an amide group, an acidimide group, a substituted oxycarbonyl group, an alkenyl group, an alkynyl group, a cyano group, and a nitro group.

Examples of the aromatic heterocyclic compound include a compound in which an aromatic ring is fused to the heterocycle even if the heterocycle itself does not exhibit aromaticity in addition to the compound in which the heterocycle itself exhibits aromaticity.

Among the aromatic heterocyclic compounds, specific examples of the compound in which the heterocycle itself exhibits aromaticity include oxadiazole, thiadiazole, thiazole, oxazol, thiophene, pyrrole, phosphole, furan, pyridine, pyrazine, pyrimidine, triazine, pyridazine, quinoline, isoquinolin, carbazole, and dibenzophosphol.

Among the aromatic heterocyclic compounds, specific examples of compounds in which the aromatic heterocycle itself does not exhibit aromaticity and the aromatic ring is fused to the heterocycle include phenoxazine, phenothiazine, dibenzoborol, dibenzosiror, and benzopyran.

The number of carbon atoms of the monovalent heterocyclic group is usually 2 to 60 and preferably 4 to 20 without including the number of carbon atoms of the substituent.

The monovalent heterocyclic group may have a substituent, and specific examples of the monovalent heterocyclic group include a thienyl group, a pyrrolyl group, a furyl group, a pyridyl group, a piperidyl group, a quinolyl group, an isoquinolyl group, a pyrimidinyl group, a triazinyl group, and a group having a substituent such as an alkyl group and an alkoxy group.

The “substituted amino group” means an amino group having a substituent. Examples of the substituent that the amino group has include an alkyl group, an aryl group, and a monovalent heterocyclic group. As the substituent, an alkyl group, an aryl group, or a monovalent heterocyclic group is preferable. The number of carbon atoms of the substituted amino group is usually 2 to 30.

Examples of the substituted amino group include a dialkylamino group such as a dimethylamino group and a diethylamino group, a diarylamino group such as a diphenylamino group, a bis(4-methylphenyl) amino group, a bis(4-tert-butylphenyl) amino group, and a bis(3,5-di-tert-butylphenyl) amino group.

The “acyl group” usually has about 2 to 20 carbon atoms, and preferably 2 to 18 carbon atoms. Specific examples of the acyl group include an acetyl group, a propionyl group, a butyryl group, an isobutyryl group, a pivaloyl group, a benzoyl group, a trifluoroacetyl group, and a pentafluorobenzoyl group.

The “imine residue” means a remaining atomic group obtained by removing one carbon atom directly bonded to a carbon atom or a nitrogen atom forming a carbon atom-nitrogen atom double bond from an imine compound. The “imine compound” means an organic compound having a carbon atom-nitrogen atom double bond in a molecule. Examples of the imine compound include aldimine, ketimine, and a compound in which the hydrogen atom bonded to the nitrogen atom constituting the carbon atom-nitrogen atom double bond in aldimine is replaced with an alkyl group or the like.

The imine residue usually has about 2 to 20 carbon atoms, and preferably 2 to 18 carbon atoms. Examples of the imine residue include groups represented by the following structural formulas.

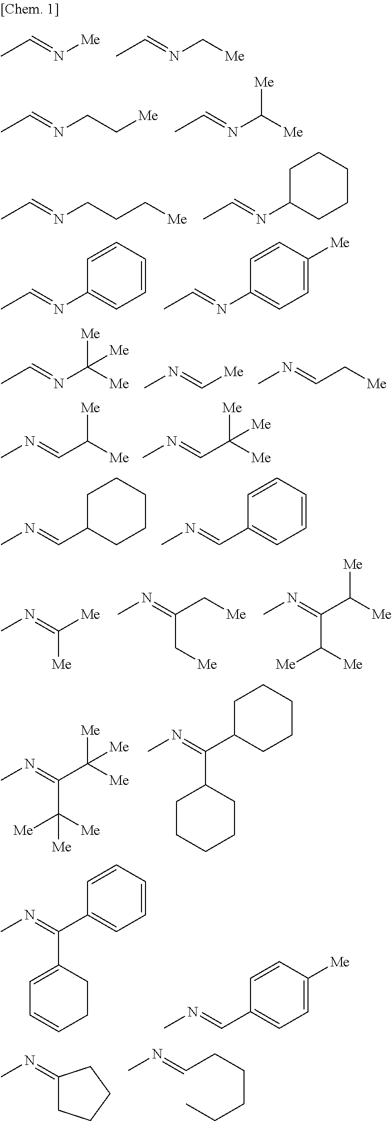

The “amide group” means a remaining atomic group obtained by removing one hydrogen atom bonded to a nitrogen atom from the amide. The number of carbon atoms of the amide group is usually about 1 to 20 and preferably 1 to 18. Specific examples of the amide group include a formamide group, an acetamido group, a propioamide group, a butyroamide group, a benzamide group, a trifluoroacetamide group, a pentafluorobenzamide group, a diformamide group, a diacetamide group, a dipropioamide group, a dibutyroamide group, a dibenzamide group, a ditrifluoroacetamide group, and a dipentafluorobenzamide group.

The “acidimide group” means a remaining atomic group obtained by removing one hydrogen atom bonded to a nitrogen atom from the acidimide. The number of carbon atoms of the acidimide group is usually about 4 to 20. Specific examples of the acidimide group include a group represented by the following structural formulas.

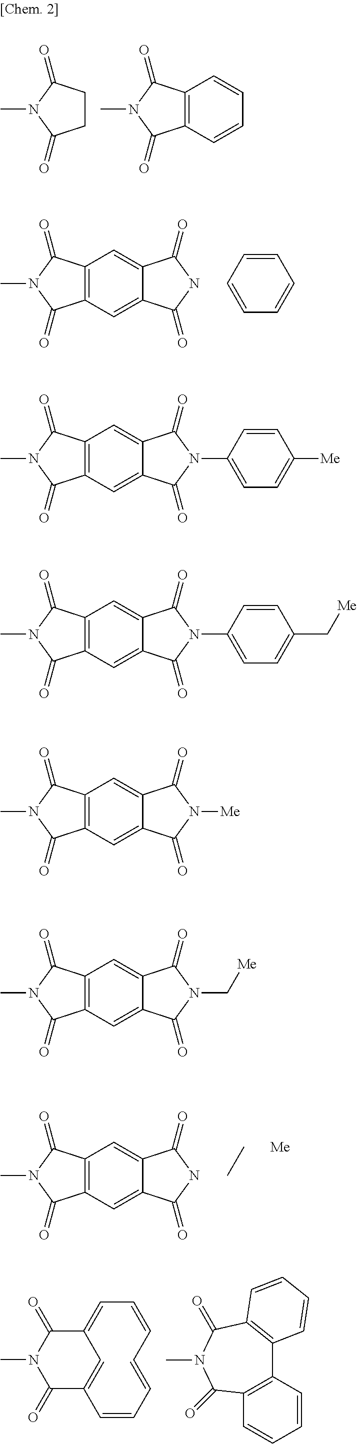

The “substituted oxycarbonyl group” means a group represented by R′—O—(C═O)—.

Here, R′ represents an alkyl group, an aryl group, an arylalkyl group, or a monovalent heterocyclic group.

The substituted oxycarbonyl group usually has about 2 to 60 carbon atoms and preferably 2 to 48 carbon atoms.

Specific examples of the substituted oxycarbonyl group include a methoxycarbonyl group, an ethoxycarbonyl group, a propoxycarbonyl group, an isopropoxycarbonyl group, a butoxycarbonyl group, an isobutoxycarbonyl group, a tert-butoxycarbonyl group, a pentyloxycarbonyl group, a hexyloxycarbonyl group, a cyclohexyloxycarbonyl group, a heptyloxycarbonyl group, an octyloxycarbonyl group, a 2-ethylhexyloxycarbonyl group, a nonyloxycarbonyl group, a decyloxycarbonyl group, a 3,7-dimethyloctyloxycarbonyl group, a dodecyloxycarbonyl group, a trifluoromethoxycarbonyl group, a pentafluoroethoxycarbonyl group, a perfluorobutoxycarbonyl group, a perfluorohexyloxycarbonyl group, a perfluorooctyloxycarbonyl group, a phenoxycarbonyl group, a naphthoxycarbonyl group, and a pyridyloxycarbonyl group.

The “alkenyl group” may be linear, branched, or cyclic. The number of carbon atoms of the linear alkenyl group is usually 2 to 30 and preferably 3 to 20 without including the number of carbon atoms of the substituent. The number of carbon atoms of the branched or cyclic alkenyl group is usually 3 to 30 and preferably 4 to 20 without including the number of carbon atoms of the substituent.

The alkenyl group may have a substituent. Specific examples of the alkenyl group include a vinyl group, a 1-propenyl group, a 2-propenyl group, a 2-butenyl group, a 3-butenyl group, a 3-pentenyl group, a 4-pentenyl group, a 1-hexenyl group, a 5-hexenyl group, a 7-octenyl group, and a group having a substituent such as an alkyl group and an alkoxy group.

The “alkynyl group” may be linear, branched, or cyclic. The number of carbon atoms of the linear alkenyl group is usually 2 to 20 and preferably 3 to 20 without including the number of carbon atoms of the substituent. The number of carbon atoms of the branched or cyclic alkenyl group is usually 4 to 30 and preferably 4 to 20 without including the number of carbon atoms of the substituent.

The alkynyl group may have a substituent. Specific examples of the alkynyl group include an ethynyl group, a 1-propynyl group, a 2-propynyl group, a 2-butynyl group, a 3-butynyl group, a 3-pentynyl group, a 4-pentynyl group, a 1-hexynyl group, a 5-hexynyl group, and a group having a substituent such as an alkyl group or an alkoxy group.

[2. Light Detecting Element]

[2.1. Overview of Light Detecting Element]

A light detecting element according to the embodiment of the present invention includes a first electrode and the second electrode, an active layer provided between the first electrode and the second electrode, and a hole transport layer provided between the second electrode and the active layer, wherein the active layer contains an organic compound, and the thickness of the active layer is 600 nm or more, and the hole transport layer contains nanoparticles of metal oxide.

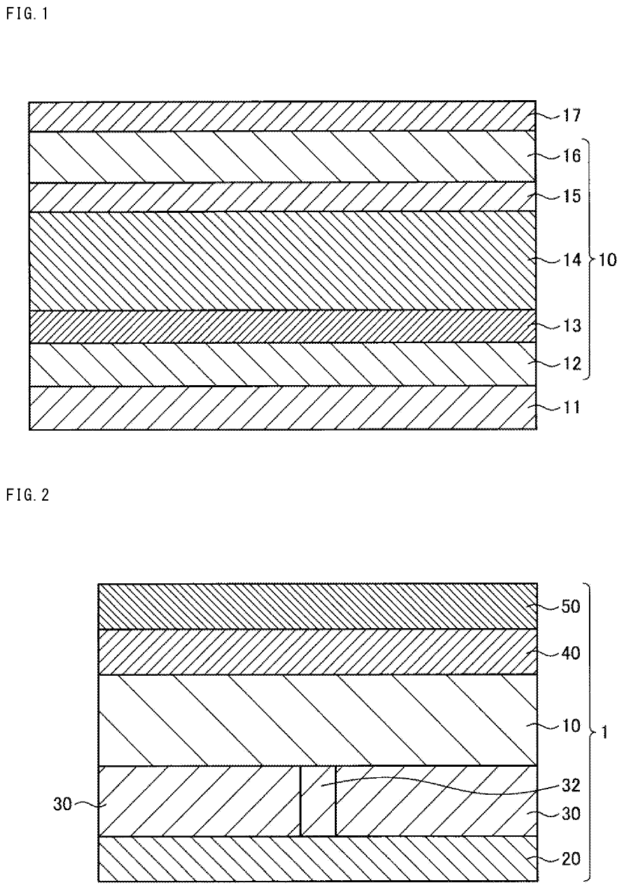

FIG. 1 is a sectional view schematically illustrating a light detecting element according to the present embodiment. As illustrated in FIG. 1, the light detecting element 10 is provided on, for example, a support substrate 11. The light detecting element 10 includes a first electrode 12, an electron transport layer 13, an active layer 14, a hole transport layer 15, and a second electrode 16 in this order. Therefore, the active layer 14 is provided between the first electrode 12 and the second electrode 16, and the hole transport layer 15 is provided between the second electrode 16 and the active layer 14. In this configuration example, a sealing substrate 17 is further provided.

The first electrode 12 is provided so as to be in contact with the support substrate 11. The electron transport layer 13 is provided so as to be in contact with the first electrode 12. The active layer 14 is provided so as to be in contact with the electron transport layer 13. The hole transport layer 15 is provided so as to be in contact with the active layer 14.

The second electrode 16 is provided so as to be in contact with the hole transport layer 15. The sealing substrate 17 is provided so as to be in contact with the second electrode 16.

[2.2. Support Substrate]

A material of the support substrate 11 is not particularly limited as long as it is a material that does not chemically change when forming a layer containing, particularly, an organic compound. Examples of the material of the support substrate 11 include glass, plastic, a polymer film, and silicon.

In a case where the support substrate 11 is opaque, it is preferable that an electrode (that is, the electrode on the side far from the opaque support substrate) on the side opposite to the electrode provided on the opaque support substrate side is a transparent or translucent electrode.

[2.3. Electrodes]

The first electrode 12 has a function as a negative electrode that allows electrons to flow out from the light detecting element 10 to an external circuit. The second electrode 16 has a function as a positive electrode that allows electrons to flow into the light detecting element 10 from an external circuit. At least one of the first electrode 12 and the second electrode 16 is preferably transparent or translucent in order to allow light to enter.

Examples of the material of the transparent or translucent electrode include a conductive metal oxide film and a translucent metal thin film. Specific examples thereof include conductive materials such as indium oxide, zinc oxide, tin oxide, and indium tin oxide (ITO), indium zinc oxide (IZO), and NESA, which are composites thereof, and gold, platinum, silver, and copper. As a transparent or translucent electrode material, ITO, IZO, and tin oxide are preferable. Further, as the electrode, a transparent conductive film using an organic compound such as polyaniline and a derivative thereof, polythiophene and a derivative thereof as a material may be used.

If one of the first electrode 12 and the second electrode 16 is transparent or translucent, the other electrode may be an electrode having low light transmittance. Examples of the material of the electrode having low light transmittance include metal and a conductive polymer. Specific examples of material of the electrode having low light transmittance include metals such as lithium, sodium, potassium, rubidium, cesium, magnesium, calcium, strontium, barium, aluminum, scandium, vanadium, zinc, yttrium, indium, cerium, samarium, europium, terbium, and ytterbium, and alloys of two or more of these, or alloys of one or more of these metals with one or more metals selected from the group consisting of gold, silver, platinum, copper, manganese, titanium, cobalt, nickel, tungsten, and tin, graphite, a graphite interlayer compound, polyaniline and a derivative thereof, and polythiophene and a derivative thereof. Examples of the alloys include a magnesium-silver alloy, a magnesium-indium alloy, a magnesium-aluminum alloy, an indium-silver alloy, a lithium-aluminum alloy, a lithium-magnesium alloy, a lithium-indium alloy, and a calcium-aluminum alloy.

As described above, in a case where the support substrate 11 is opaque, it is preferable that an electrode (that is, the electrode on the side far from the support substrate) on the side opposite to the electrode provided on the opaque support substrate 11 side is a transparent or translucent electrode.

That is, in the present embodiment, in the case where the support substrate 11 is opaque, it is preferable that the second electrode 16 is a transparent or translucent electrode.

[2.4. Electron Transport Layer]

The electron transport layer 13 has a function of transporting the electrons generated in the active layer 14 to the first electrode 12. The electron transport layer 13 may have a function as a hole block layer that prevents the holes generated in the active layer 14 from being transported to the first electrode 12.

The electron transport layer 13 usually contains an electron transport material. Examples of the electron transport material include polyalkyleneimine and a derivative thereof, a polymer compound having a fluorene structure, and metal oxide.

Examples of polyalkyleneimine and a derivative thereof include alkyleneimine having 2 to 8 carbon atoms such as ethyleneimine, propyleneimine, butyleneimine, dimethylethyleneimine, pentyleneimine, hexyleneimine, heptyleneimine, and octyleneimine, particularly, polymers obtained by polymerizing one or two or more of alkyleneimines having 2 to 4 carbon atoms by a common method, and a polymers that are chemically modified by reacting them with various compounds. More specifically, for example, polyethyleneimine (PEI) and polyethyleneimine ethoxylate (PEIE: ethoxylated polyethyleneimine) can be mentioned. Polyethyleneimine ethoxylate is a modified product containing polyalkyleneimine as a main chain and ethylene oxide added to a nitrogen atom in the main chain.

Examples of the polymer compound having a fluorene structure include poly[(9,9-bis(3′-(N,N-dimethylamino) propyl)-2,7-fluorene)-ortho-2,7-(9,9′-dioctylfluorene)] (PFN).

Examples of the metal oxide include zinc oxide, gallium-doped zinc oxide, aluminum-doped zinc oxide, titanium oxide, and niobium oxide.

Examples of other electron transport materials include poly(4-vinylphenol) and perylene diimide.

[2.5. Active Layer]

[Configuration of Active Layer]

The active layer 14 contains an organic compound, preferably a polymer compound, more preferably a conjugated polymer compound, and usually contains a p-type semiconductor material (electron-donating compound) and an n-type semiconductor material (electron-accepting compound). Examples of the p-type semiconductor materials and n-type semiconductor materials and suitable examples will be described later. Whether the material to be contained is the p-type semiconductor material or the n-type semiconductor material can be relatively determined from a HOMO or a LUMO energy level of the selected compound.

Here, the conjugated polymer compound means a polymer compound having a structure in which n electrons are delocalized due to overlapping p orbitals in the main chain, and examples thereof include (1) a polymer compounds having a structure in which a double bond and a single bond are alternately arranged, (2) a polymer compound having a structure in which a double bond and a single bond are arranged via a nitrogen atom, and (3) a polymer compound having a structure in which a double bond and a single bonds are alternatively arranged and a structure in which a double bond and a single bond are arranged via a nitrogen atom.

The active layer 14 preferably has a structure in which a p-type semiconductor material phase and an n-type semiconductor material phase are separated from each other, that is, a so-called bulk heterojunction type structure.

[Thickness of Active Layer]

A thickness of the active layer 14 is usually 600 nm or more, preferably 700 nm or more, more preferably 800 nm or more, and further preferably 1000 nm or more from the viewpoint of reducing the dark current, and it is preferably 5000 nm or less, more preferably 3000 nm or less, and further preferably 1500 nm or less from the viewpoint of improving the external quantum efficiency.

The thickness of the active layer 14 can be adjusted by a known method in the related art. For example, in a case where the active layer 14 is formed by a coating method, the thickness of the active layer 14 may be adjusted by adjusting the concentration (solid content concentration) of the components excluding the solvent contained in the coating liquid to be used, coating conditions, and the like.

Specifically, for example, in a case where the coating method is the knife coating method, the thickness of the active layer 14 is adjusted by adjusting the coating speed and/or adjusting a gap between the coating surface and the knife.

Further, for example, the active layer 14 can be thickened by increasing the solid content concentration of the coating liquid, and the thickness of the active layer 14 can be reduced by decreasing the solid content concentration.

The thickness of the active layer can be measured with a stylus type film thickness meter (“Surfcorder ET-200” available from Kosaka Laboratory Ltd.).

[Material of Active Layer]

(p-Type Semiconductor Material)

The p-type semiconductor material that can be contained in the active layer may be a low molecular weight compound or a polymer compound. The p-type semiconductor material is preferably a polymer compound, and more preferably a conjugated polymer compound.

Examples of the p-type semiconductor material comprising low molecular weight compound include phthalocyanine, metallic phthalocyanine, porphyrin, metallic porphyrin, oligothiophene, tetracene, pentacene, and rubrene.

In a case where the p-type semiconductor material is a polymer compound, the polymer compound preferably has a predetermined polystyrene-equivalent weight average molecular weight.

Here, the polystyrene-equivalent weight average molecular weight means a weight average molecular weight calculated using a standard polystyrene sample using gel permeation chromatography (GPC).

The polystyrene-equivalent weight average molecular weight of the p-type semiconductor material is preferably 20000 or more and 200000 or less, more preferably 30000 or more and 180000 or less, and further preferably 40000 or more and 150000 or less from the viewpoint of solubility in a solvent.

Examples of the p-type semiconductor material which is a conjugated polymer compound include polyvinylcarbazole and a derivative thereof, polysilane and a derivative thereof, a polysiloxane derivative having an aromatic amine structure in a side chain or main chain, polyaniline and a derivative thereof, polythiophene and a derivative thereof, polypyrrole and a derivative thereof, polyphenylene vinylene and a derivative thereof, polythienylene vinylene and a derivative thereof, and polyfluorene and a derivative thereof.

The p-type semiconductor material, which is the conjugated polymer compound, is preferably a polymer compound containing a constituent unit having a thiophene skeleton.

The p-type semiconductor material is preferably a polymer compound containing a constituent unit represented by the following Formula (I) and/or a constituent unit represented by the following Formula (II), and is more preferably a conjugated polymer compound containing a constituent unit represented by the following Formula (I) and/or a constituent unit represented by the following Formula (II).

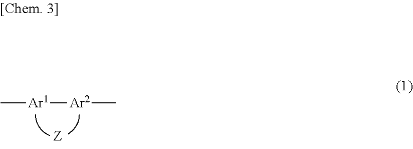

In Formula (I), Ar¹ and Ar² represent trivalent aromatic heterocyclic groups, and Z represents groups represented by the following Formulas (Z-1) to (Z-7).

[Chem. 4]

—Ar³— (II)

In Formula (II), Ara represents a divalent aromatic heterocyclic group.

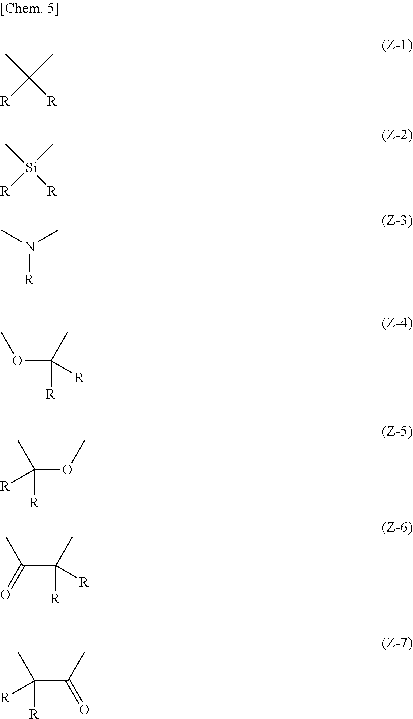

In Formulas (Z-1) to (Z-7), R is a hydrogen atom, a halogen atom, an alkyl group, an aryl group, an alkoxy group, an aryloxy group, an alkylthio group, an arylthio group, a monovalent heterocyclic group, a substituted amino group, an acyl group, an imine residue, an amide group, an acidimide group, a substituted oxycarbonyl group, an alkenyl group, an alkynyl group, a cyano group, or a nitro group. In a case where there are two Rs in each of the Formulas (Z-1) to (Z-7), the two Rs may be the same or different from each other.

The constituent unit represented by Formula (I) is preferably the constituent unit represented by the following Formula (I-1).

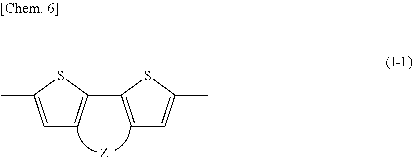

In Formula (I-1), Z has the same meaning as described above.

Examples of the constituent unit represented by Formula (I-1) include constituent units represented by the following Formulas (501) to (505).

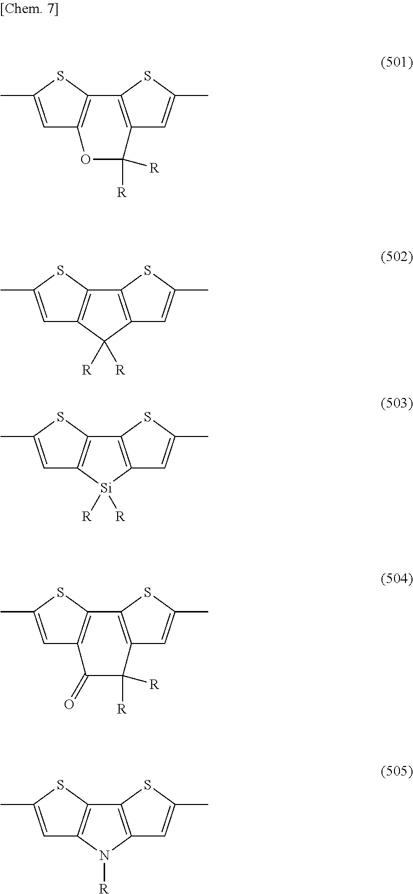

In the Formulas (501) to (505), R has the same meaning as described above. In a case where there are two Rs, the two Rs may be the same or different from each other.

The number of carbon atoms of the divalent aromatic heterocyclic group represented by Ara is usually 2 to 60, preferably 4 to 60, and more preferably 4 to 20. The divalent aromatic heterocyclic group represented by Ar³ may have a substituent. Examples of substituents that the divalent aromatic heterocyclic group represented by Ar³ may have include a halogen atom, an alkyl group, an aryl group, an alkoxy group, an aryloxy group, an alkylthio group, an arylthio group, a monovalent heterocyclic group, a substituted amino group, an acyl group, an imine residue, an amide group, an acidimide group, a substituted oxycarbonyl group, an alkenyl group, an alkynyl group, a cyano group, and a nitro group.

Examples of the divalent aromatic heterocyclic group represented by Ar³ include groups represented by the following Formulas (101) to (185).

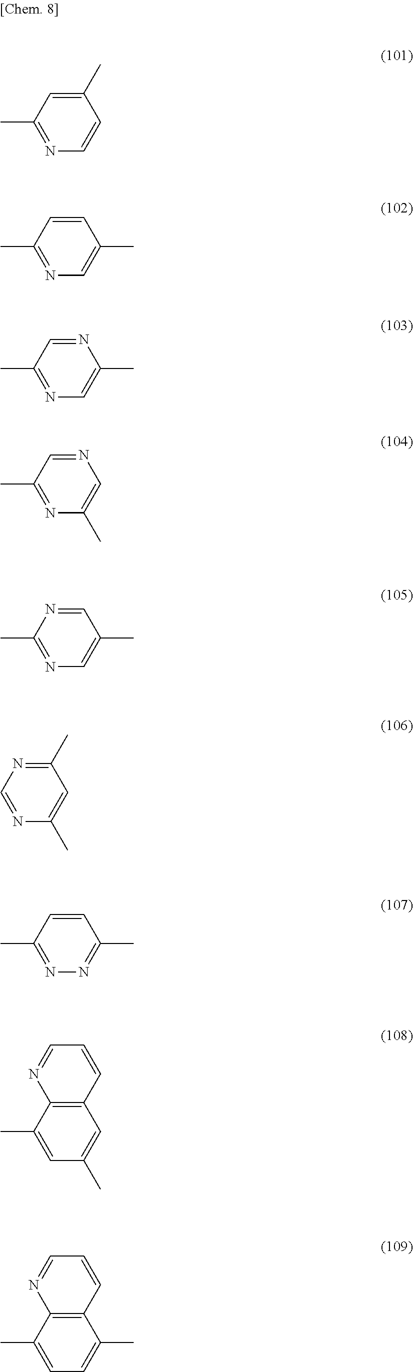

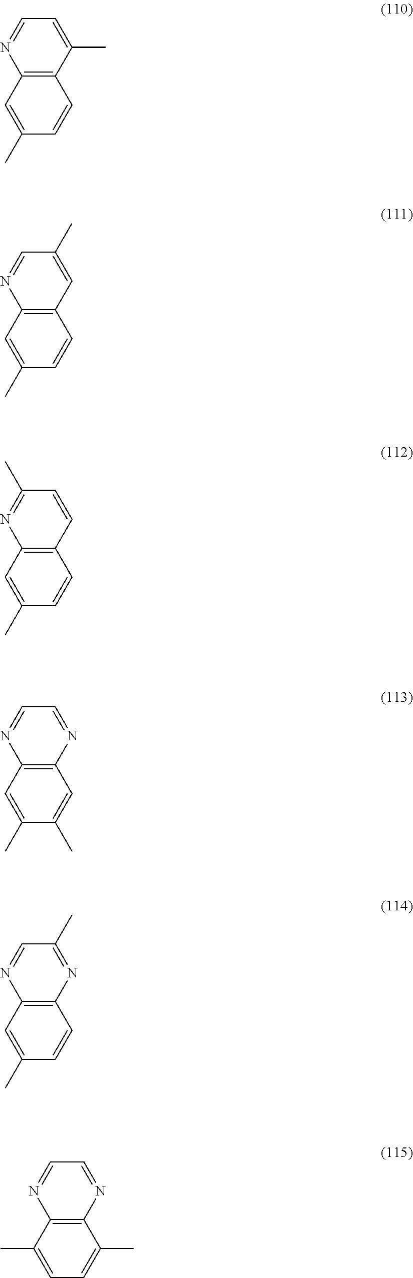

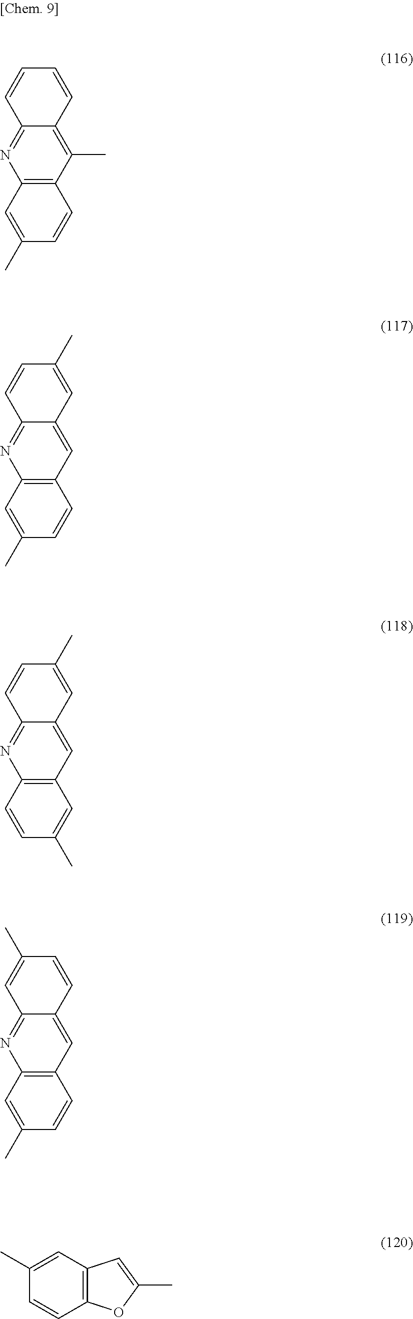

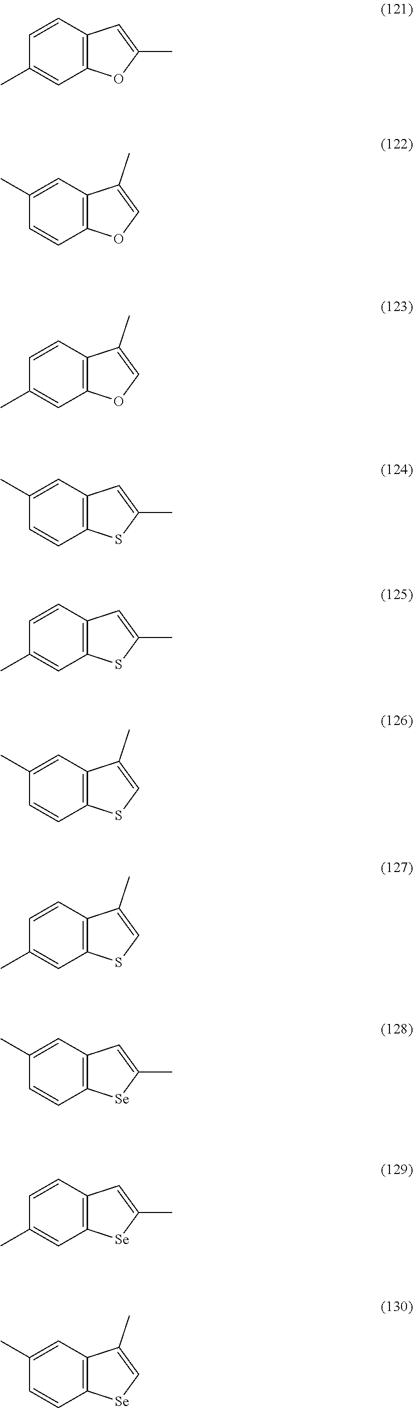

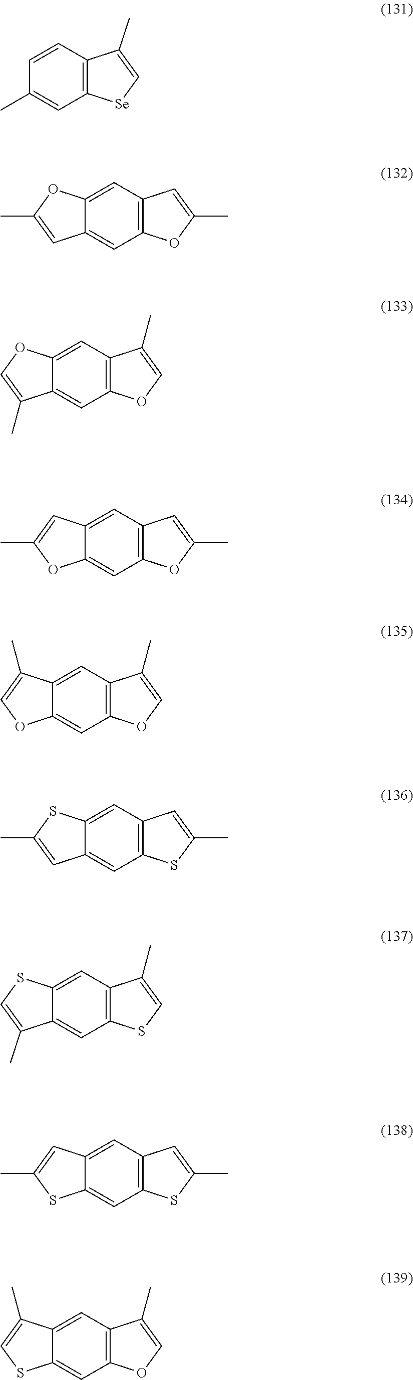

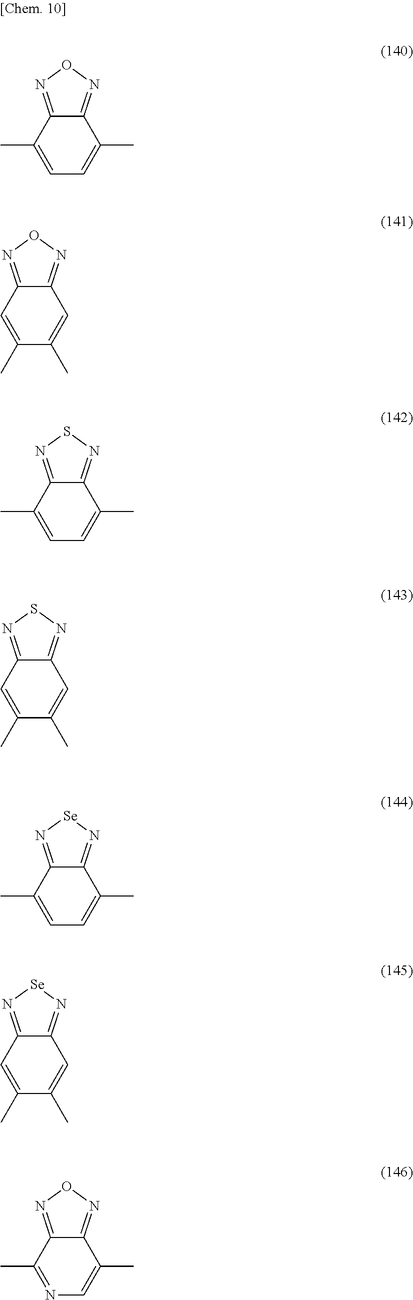

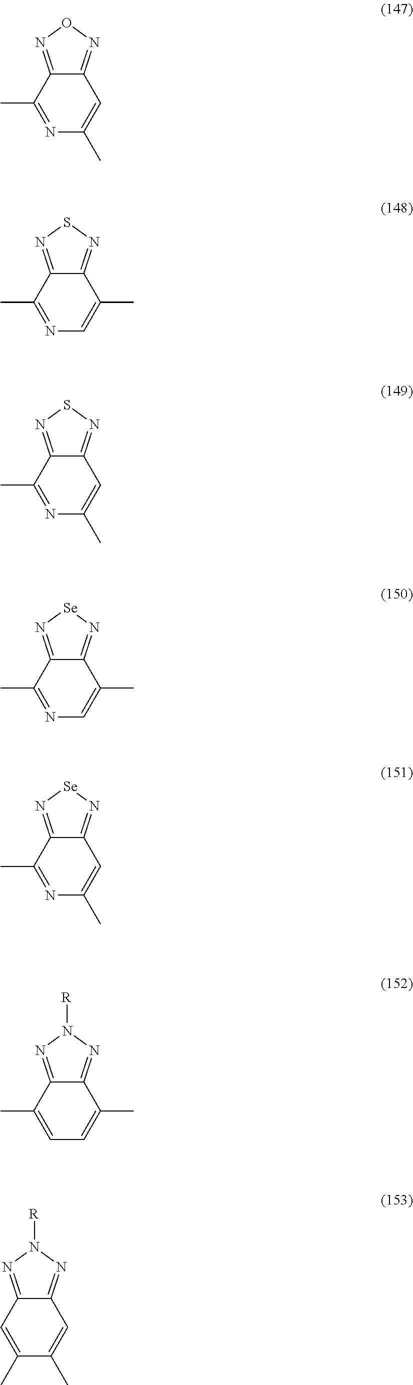

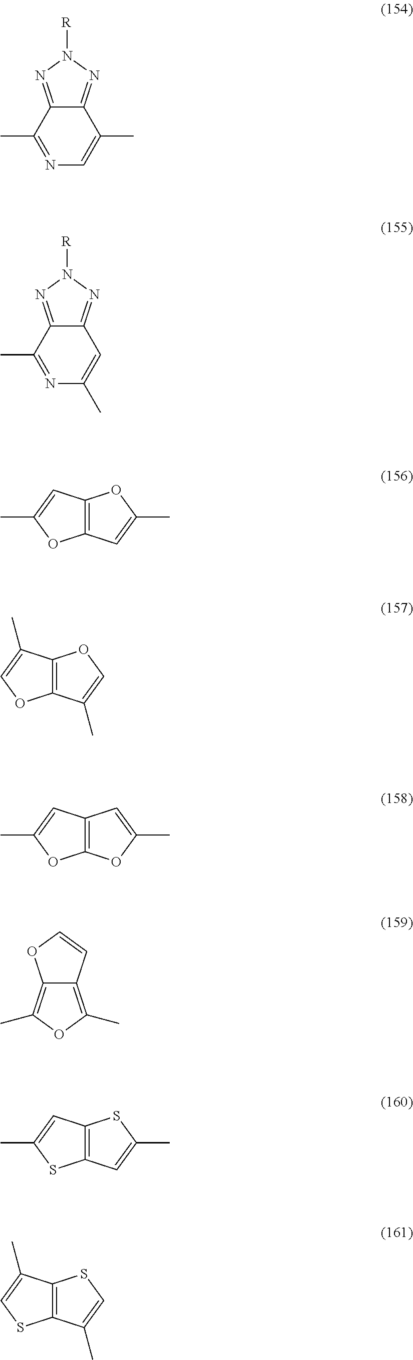

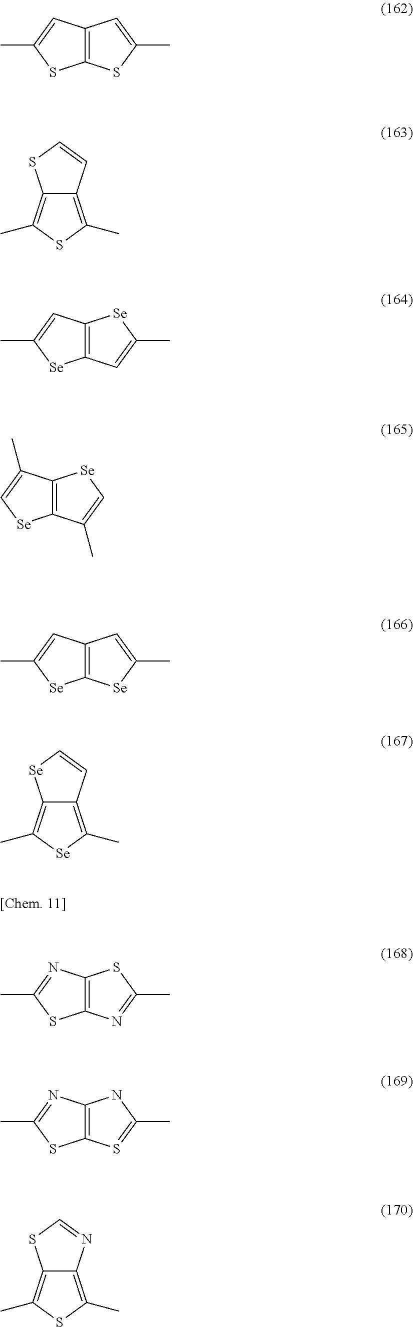

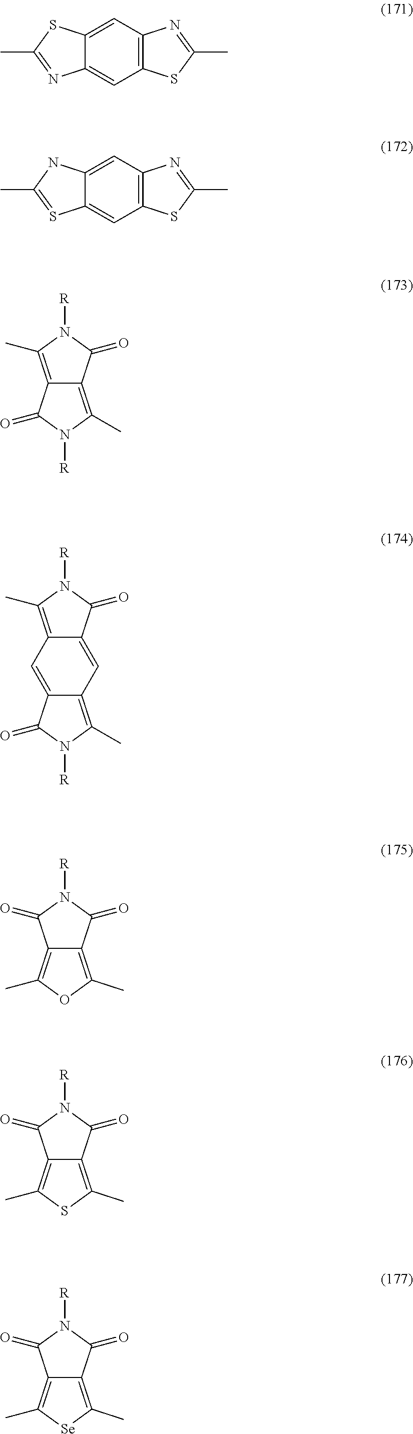

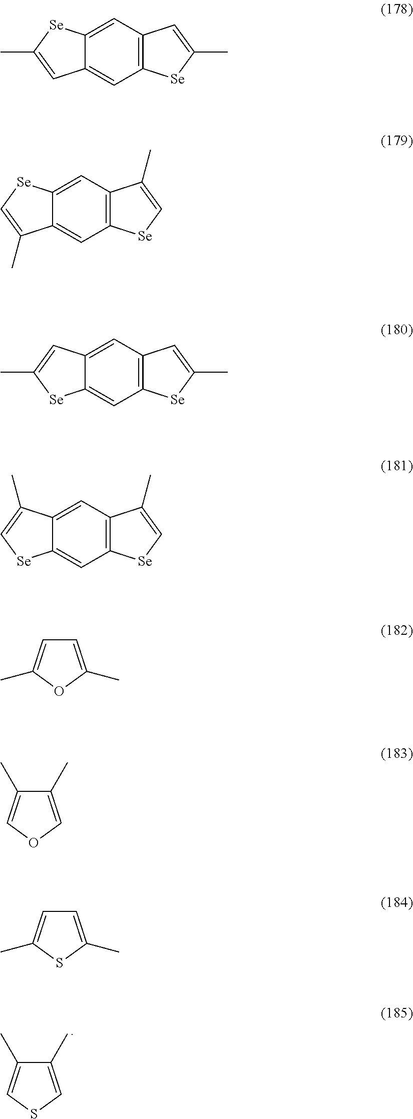

In the Formulas (101) to (185), R has the same meaning as described above. In a case where there are a plurality of Rs, the plurality of Rs may be the same or different from each other.

As the constituent unit represented by the Formula (II), constituent units represented by the following Formulas (II-1) to (II-6) are preferable.

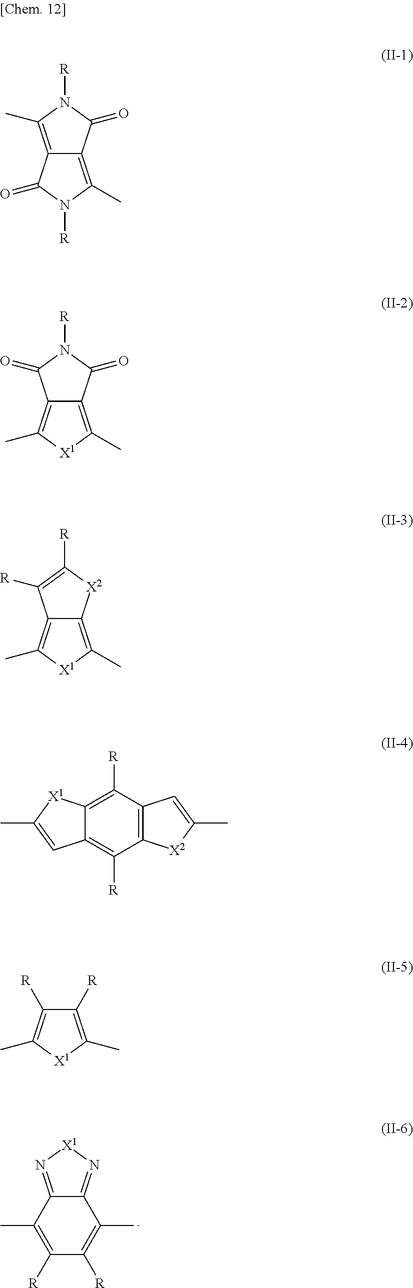

In Formulas (II-1) to (II-6), X¹ and X² each independently represent an oxygen atom or a sulfur atom, and R has the same meaning as described above. In a case where there are a plurality of Rs, the plurality of Rs may be the same or different from each other.

From the viewpoint of availability of the raw material compound, it is preferable that both X¹ and X² in the Formulas (II-1) to (II-6) are sulfur atoms.

The polymer compound which is the p-type semiconductor material may have two or more kinds of constituent units of Formula (I), and may have two or more kinds of constituent units of Formula (II).

In order to improve the solubility in the solvent, the polymer compound which is the p-type semiconductor material may have a constituent unit represented by the following Formula (III).

[Chem. 13]

—Ar⁴— (III)

In Formula (III), Ar⁴ represents an arylene group.

The arylene group represented by Ar⁴ means a remaining atomic group obtained by removing two hydrogen atoms from the aromatic hydrocarbon which may have a substituent. Aromatic hydrocarbons also include compounds in which two or more selected from the group consisting of a compound having a fused ring, an independent benzene ring, and a fused ring are bonded directly or via a divalent group such as a vinylene group.

Examples of the substituents that the aromatic hydrocarbon may have include the same substituents as those exemplified as the substituents that the heterocyclic compound may have.

The number of carbon atoms in a portion of the arylene group excluding the substituent is usually 6 to 60, and preferably 6 to 20. The number of carbon atoms of the arylene group including the substituent is usually about 6 to 100.

Examples of the arylene group include a phenylene group (for example, the following Formulas 1 to 3), a naphthalene-diyl group (for example, the following Formulas 4 to 13), an anthracene-diyl group (for example, the following Formulas 14 to 19), a biphenyl-diyl group (for example, the following Formulas 20 to 25), a terphenyl-diyl group (for example, the following Formulas 26 to 28), a fused ring compound group (for example, the following Formulas 29 to 35), a fluorene-diyl group (for example, the following Formulas 36 to 38), and a benzofluorene-diyl group (for example, the following Formulas 39 to 46).

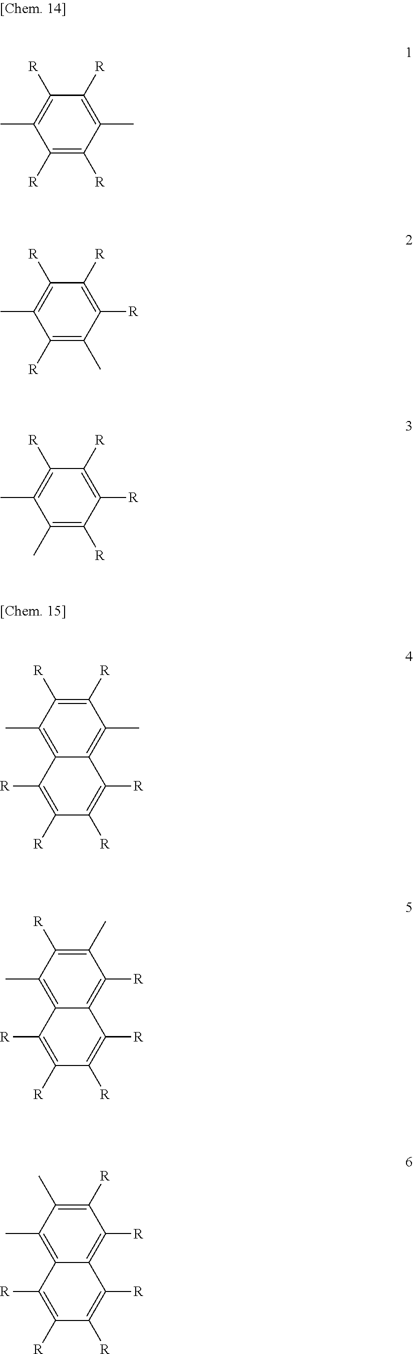

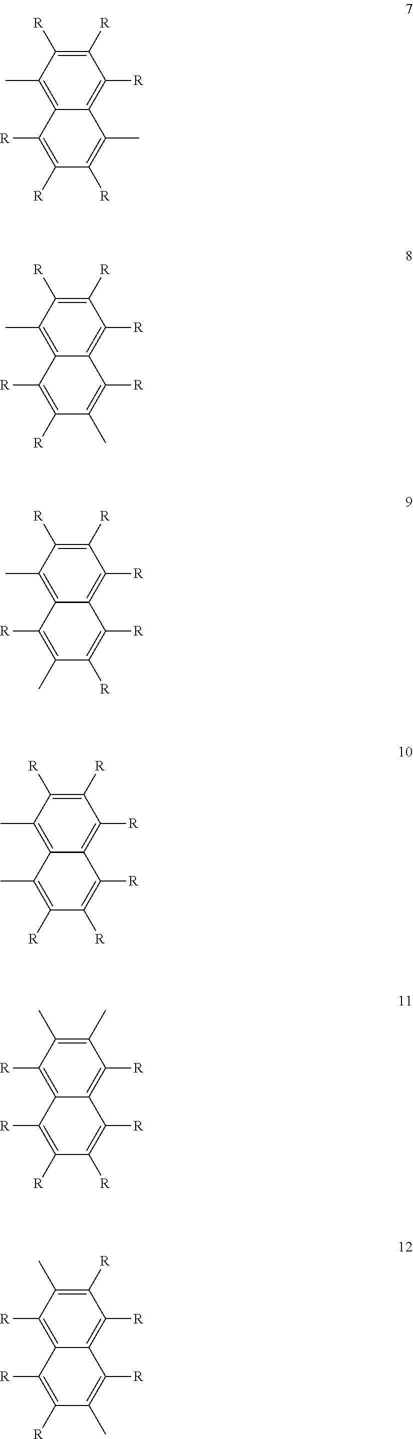

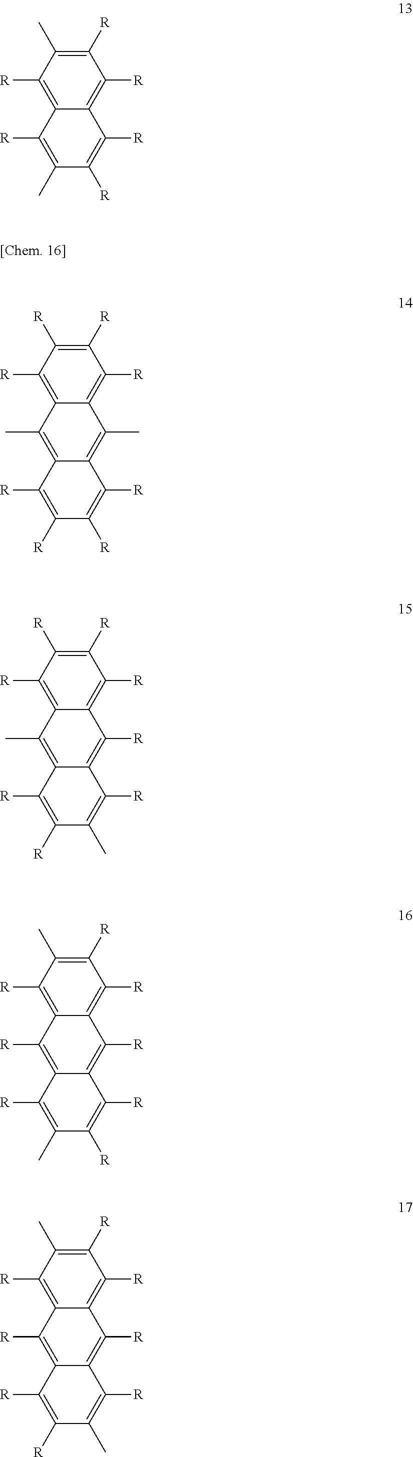

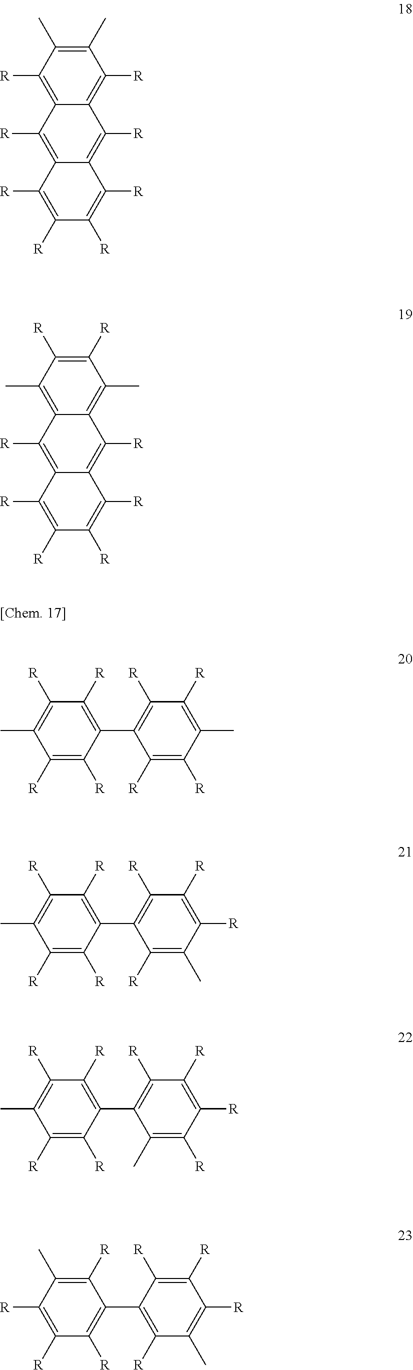

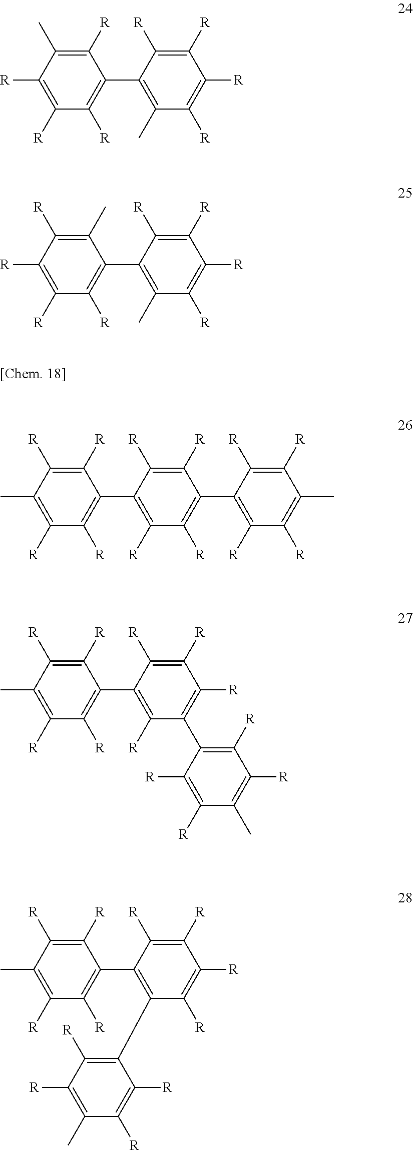

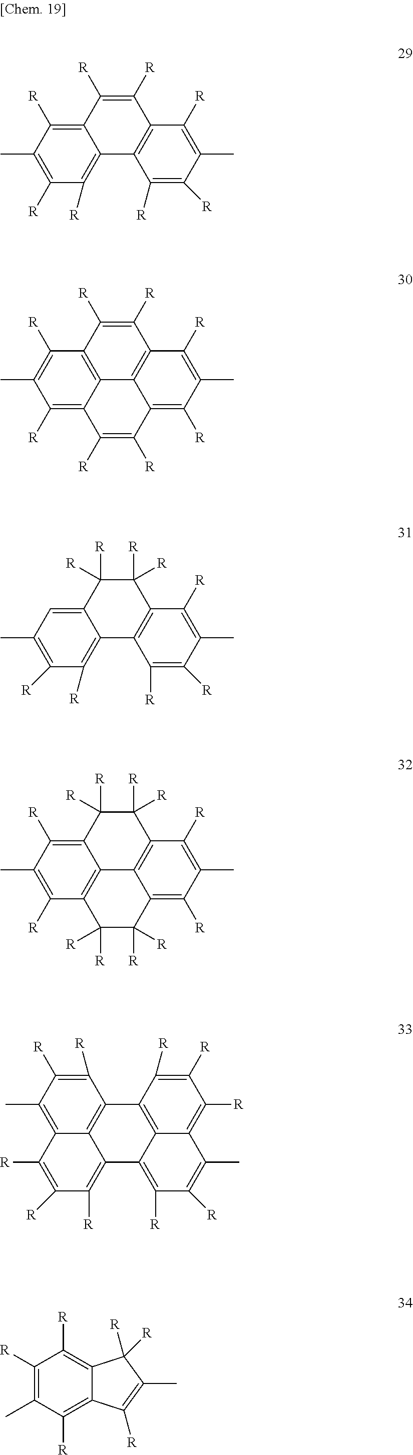

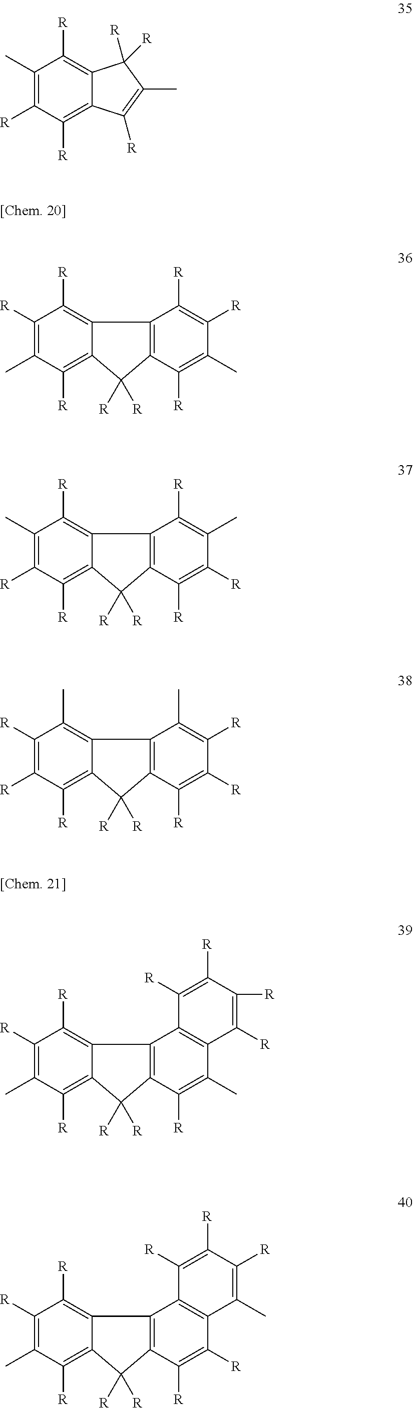

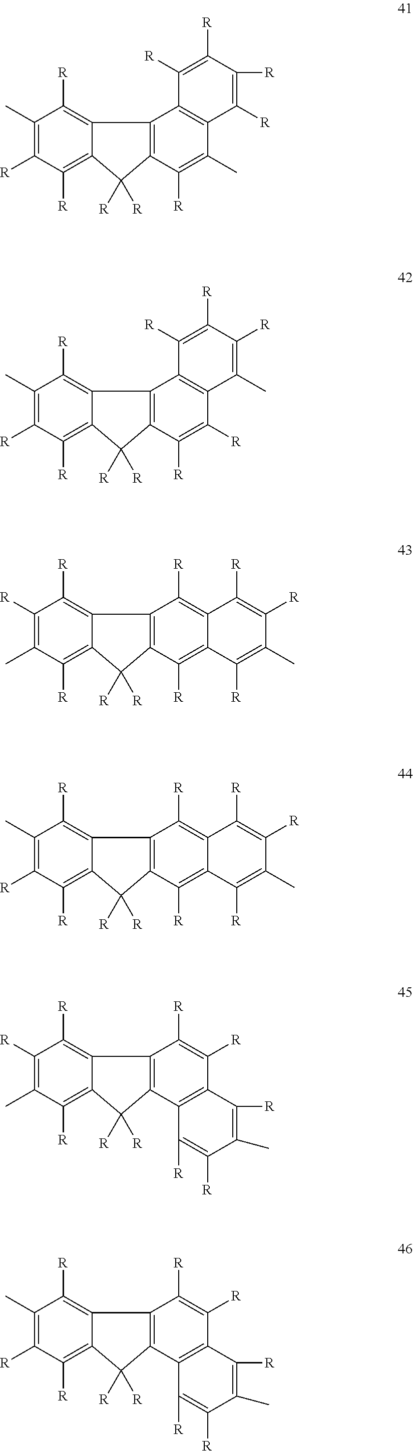

The constituent unit constituting the polymer compound, which is a p-type semiconductor material, may be a constituent unit in which two or more types selected from the constituent unit represented by Formula (I), the constituent unit represented by Formula (II), and the constituent unit represented by Formula (III) are combined and connected to each other.

In a case where the polymer compound as a p-type semiconductor material has the constituent unit represented by Formula (I) and/or the constituent unit represented by Formula (II), assuming that the amount of all the constituent units contained in the polymer compound is 100 mol %, the total amount of the constituent unit represented by Formula (I) and the constituent unit represented by Formula (II) is usually 20 to 100 mol %, and in order to improve charge transport properties as a p-type semiconductor material, it is preferably 40 to 100 mol %, and more preferably 50 to 100 mol %.

Specific examples of the polymer compound as a p-type semiconductor material include polymer compounds represented by the following Formulas P-1 to P-6.

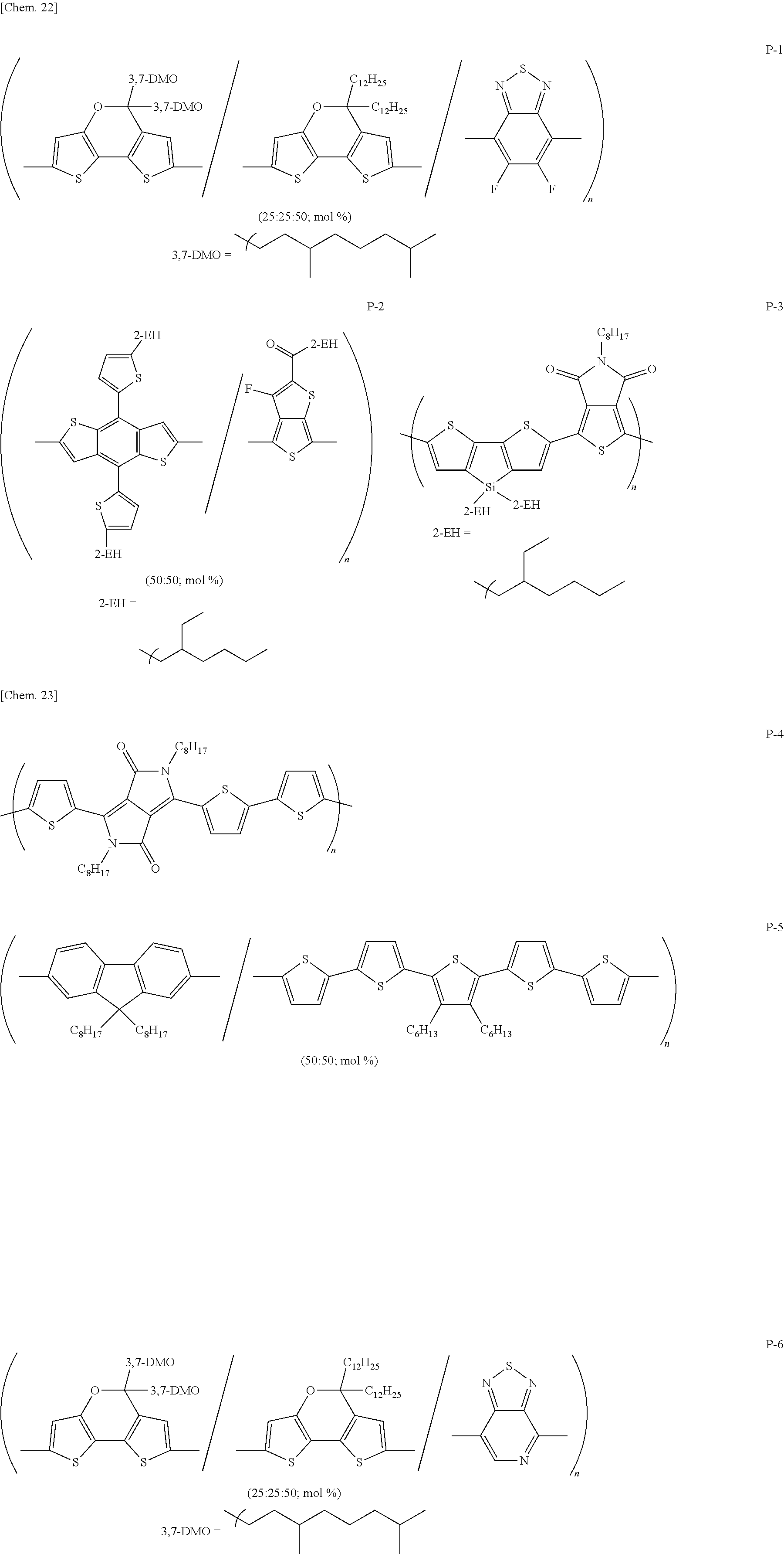

The active layer may contain only one type of p-type semiconductor material, or may contain two or more types in optional ratio combination.

(n-Type Semiconductor Material)

Examples of the n-type semiconductor material that can be contained in the active layer include polyvinylcarbazole and a derivative thereof, polysilane and a derivative thereof, a polysiloxane derivative having an aromatic amine structure in a side chain or main chain, polyaniline and a derivative thereof, polythiophene and a derivative thereof, polypyrrole and a derivative thereof, polyphenylene vinylene and a derivative thereof, polythienylene vinylene and a derivative thereof, polyquinoxaline and a derivative thereof, polyquinoxaline and a derivative thereof, and polyfluorene and a derivative thereof.

The n-type semiconductor material is preferably one or more selected from fullerene and a fullerene derivative, and more preferably a fullerene derivative.

Examples of the fullerene include C60 fullerene, C₇₀ fullerene, C₇₆ fullerene, C₇₈ fullerene, and C₈₄ fullerene. Examples of the fullerene derivative include a derivative of these fullerenes. The fullerene derivative means a compound in which at least a part of fullerene is modified.

Examples of fullerene derivatives include compounds represented by the following Formulas (N-1) to (N-4).

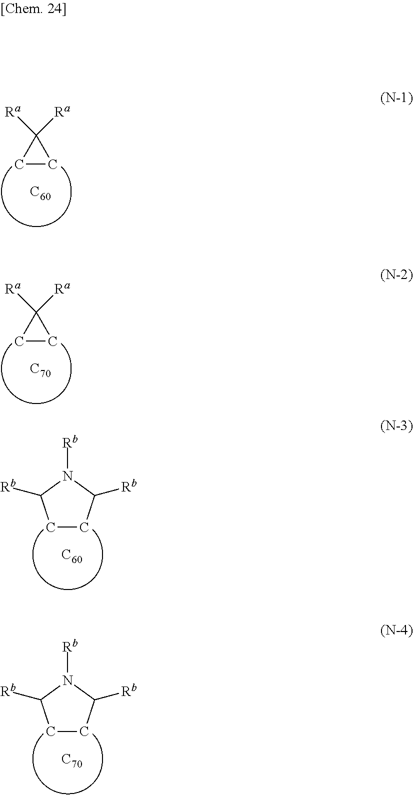

In Formulas (N-1) to (N-4), R^{a} represents an alkyl group, an aryl group, a monovalent heterocyclic group, or a group having an ester structure. A plurality of R^{a}s may be the same or different from each other.

R^{b} represents an alkyl group or an aryl group. A plurality of R^{b}s may be the same or different from each other.

Examples of the group having an ester structure represented by R^{a} include a group represented by the following Formula (19).

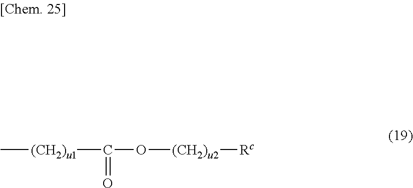

In Formula (19), u1 represents an integer from 1 to 6. u2 represents an integer from 0 to 6. R^{c} represents an alkyl group, an aryl group, or a monovalent heterocyclic group.

Examples of C₆₀ fullerene derivatives include the following compounds.

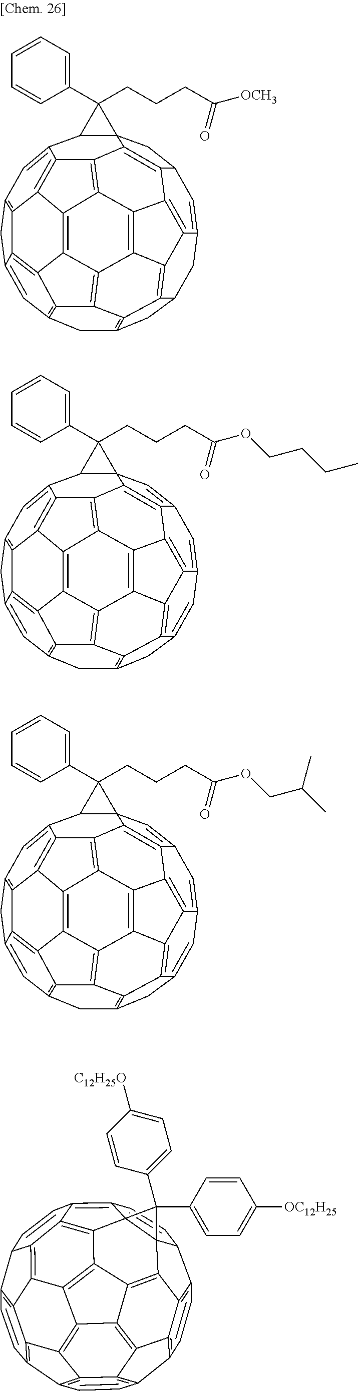

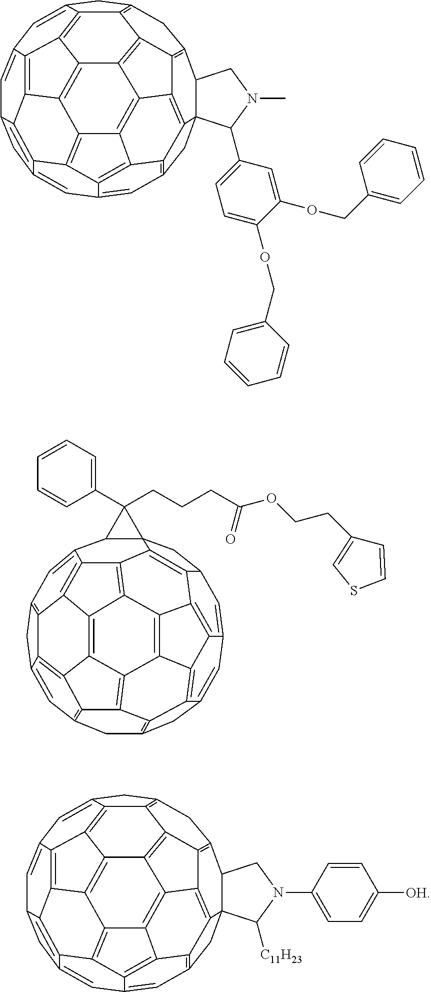

Specific examples of the C₆₀ fullerene derivative include [6,6]-phenyl-C61 butyric acid methyl ester (C60PCBM, [6,6]-Phenyl-C61 butyric acid methyl ester), and [6,6]-thienyl-C61 butyric acid methyl ester ([6,6]-Thienyl C61 butyric acid methyl ester)

The active layer may contain only one type of n-type semiconductor material, or may contain two or more types in optional ratio combination.

[2.6. Hole Transport Layer]

The hole transport layer 15 has a function of transporting the holes generated in the active layer 14 to the second electrode 16. The hole transport layer 15 may have a function as an electron block layer that prevents the electrons generated in the active layer 14 from being transported to the second electrode 16.

The hole transport layer 15 contains nanoparticles of metal oxide.

(Nanoparticles)

In the present specification, the term “nanoparticles” refers to particles having an average particle size smaller than 1 μm.

The average particle size of nanoparticles is usually larger than 0 nm. The hole transport layer 15 can reduce the dark current by containing nanoparticles of the metal oxide.

From the viewpoint of smoothing the hole transport layer, the average particle size of the nanoparticles of the metal oxide is preferably 50 nm or less, and more preferably 30 nm or less. The lower limit of the average particle size of the nanoparticles of the metal oxide is preferably 5 nm or more.

From the viewpoint of making the hole transport layer thin and dense to enhance the hole transport property, the average particle size of the nanoparticles of the metal oxide is preferably 5 nm or more and 30 nm or less.

Here, the average particle size of the nanoparticles is a value measured by a dynamic light scattering (DLS) method.

(Metal Oxide)

The metal oxide may be either a single metal element oxide or a composite oxide composed of a plurality of metal element oxides. Preferably, the metal oxide contains at least one selected from the group consisting of a molybdenum atom, a tungsten atom, and a nickel atom.

Examples of the metal oxides include molybdenum oxide (such as MoO₃), vanadium oxide (such as V₂O₅), tungsten oxide (such as WO₃), and nickel oxide (such as NiO), preferably is one or more selected from the group consisting of molybdenum oxide, tungsten oxide, and nickel oxide, and more preferably molybdenum oxide, tungsten oxide, or nickel oxide.

MoO₃ is preferable as the molybdenum oxide. WO₃ is preferable as the tungsten oxide. NiO is preferable as nickel oxide.

The hole transport layer 15 may contain only one type of metal oxide in the form of nanoparticles, or may contain two or more types of metal oxides in the form of nanoparticles. In a case where the hole transport layer 15 contains two or more kinds of metal oxides, the nanoparticles of each metal oxide may have different average particle sizes from each other.

The nanoparticles of the metal oxide may be treated with a surface treatment agent in order to improve the dispersibility in the solvent.

Examples of the surface treatment agents include a silane coupling agent.

The hole transport layer 15 may contain a hole transport material other than the nanoparticles of the metal oxide.

Examples of hole transport materials other than nanoparticles of the metal oxide include polythiophene and a derivative thereof, an aromatic amine compound, a polymer compound containing a constituent unit having an aromatic amine residue.

The hole transport material other than the nanoparticles of the metal oxide, which can be contained in the hole transport layer 15, is, for example, preferably 5% by weight or less, more preferably 1 by weight, usually 0% by weight or more, and may be 0% by weight, based on the weight of the nanoparticles of metal oxide.

The thickness of the hole transport layer can be optionally set, and may be, for example, 5 nm or more, 10 nm or more, or 50 nm or more, and may be 200 nm or less, 100 nm or less, or 80 nm or less.

[2.7. Sealing Substrate]

The light detecting element 10 may be sealed by a sealing substrate 17. Examples of the sealing member include a combination of cover glass having a recess and a sealing substrate.

The sealing substrate may have a layer structure of one or more layers. Therefore, as an example of the sealing substrate, a layer structure such as a gas barrier layer and a gas barrier film can be further exemplified.

The layer structure of the sealing substrate is preferably formed of a material having a property of blocking water (water vapor barrier property) or a property of blocking oxygen (oxygen barrier property). Examples of suitable materials as materials having a layer structure include organic materials such as resins of polyethylene trifluoride, polychlorotrifluoroethylene (PCTFE), polyimide, polycarbonate, polyethylene terephthalate, alicyclic polyolefin, and an ethylene-vinyl alcohol copolymer; and inorganic materials such as silicon oxide, silicon nitride, aluminum oxide, and diamond-like carbon.

[2.8. Another Embodiment of Light Detecting Element]

The light detecting element of the above embodiment includes a support substrate, a first electrode, an electron transport layer, an active layer, a hole transport layer, a second electrode, and a sealing substrate in this order; however, the light detecting element may include the support substrate, the second electrode, the hole transport layer, the active layer, the electron transport layer, the first electrode, and the sealing substrate in this order. Further, the light detecting element may not include an electron transport layer, and the active layer may be in contact with the first electrode. Further, the light detecting element may not include a support substrate and may not include a sealing substrate.

[3. Method for Producing Light Detecting Element]

The method for producing the light detecting element according to the present invention is not particularly limited. The light detecting element according to the present invention can be produced by a suitable forming method by using a material selected for forming each component.

For example, the light detecting element according to one embodiment of the present invention, which includes a support substrate, a first electrode, an electron transport layer, an active layer, a hole transport layer, and a second electrode in this order, can be produced by a producing method including the following steps this order.

Step (I) Step of preparing a support substrate provided with the first electrode

Step (II) Step of forming an electron transport layer

Step (III) Step of forming an active layer

Step (VI) Step of forming a hole transport layer containing nanoparticles of metal oxide

Step (V) Step of forming a second electrode

Hereinafter, each step will be described in order.

[Step (I)]

In this step, a support substrate provided with a first electrode is prepared. A method of providing the first electrode on the support substrate is not particularly limited. The first electrode can be formed, for example, by laminating the above-exemplified material on the support substrate made of the materials as described above by a vacuum deposition method, a sputtering method, an ion plating method, a plating method, a coating method, or the like.

Further, a support substrate provided with a thin film formed of the material of the electrode as described above is available from the market, and if necessary, the first electrode is formed by patterning a conductive thin film, thereby preparing a support substrate provided with the first electrode.

[Step (II)]

In this step, an electron transport layer is formed. Since the electron transport layer can be easily formed at low cost, it is preferable to form the electron transport layer by a coating method using a coating liquid.

Examples of the coating method for forming an electron transport layer include a slit coating method, a knife coating method, a spin coating method, a micro gravure coating method, a gravure coating method, a bar coating method, an ink jet coating method, a nozzle coating method, a capillary casting method, a casting method, a roll coating method, a wire bar coating method, a dip coating method, a spray coating method, a screen printing method, a flexographic printing method, an offset printing method, and a dispenser printing method. Among them, a slit coating method, a knife coater method, a spin coating method, a micro gravure coating method, a gravure coating method, a bar coating method, an ink jet printing method, a nozzle coating method, or a capillary coating method is preferable, and the slit coating method, the knife coating method, the spin coating method, the bar coat method, or the capillary coat method is more preferable, and the slit coat method, the knife coating method, or the spin coating method is further preferable.

The coating liquid for forming an electron transport layer by the coating method usually contains an electron transport material and a solvent. Examples of the electron transport material that can be contained in the coating liquid are the same as those described above as the electron transport material that can be contained in the electron transport layer.

Examples of the solvent that can be contained in the coating liquid for forming an electron transport layer include water, an alcohol solvent, a ketone solvent, and a hydrocarbon solvent. Specific examples of the alcohol solvent include methanol, ethanol, isopropanol, butanol, ethylene glycol, propylene glycol, butoxyethanol, and methoxybutanol. Specific examples of the ketone solvent include acetone, methyl ethyl ketone, methyl isobutyl ketone, 2-heptanone, and cyclohexanone. Specific examples of the hydrocarbon solvent include n-pentane, cyclohexane, n-hexane, benzene, toluene, xylene, tetralin, chlorobenzene, and orthodichlorobenzene. The coating liquid may contain one kind of solvent alone, may contain two or more kinds of solvents, and may contain two or more kinds of the above-exemplified solvents.

The coating liquid used in the coating method for forming the electron transport layer may be a dispersion liquid such as an emulsion (emulsion liquid) or a suspension (suspension).

After applying the coating liquid to the coating target to form a coating film, the solvent is usually removed from the coating film. Examples of the method for removing the solvent include a method for direct heating using a hot plate, and a drying method such as a hot air drying method, an infrared heating drying method, a flash lamp annealing drying method, and a vacuum drying method.

[Step (III)]

In this step, an active layer is formed. Since the active layer can be easily formed at low cost, it is preferable to form the active layer by a coating method using a coating liquid.

Examples and preferable examples of the coating method for forming the active layer are the same as the above-described examples and preferable examples of the coating method for forming the electron transport layer. As the coating method for forming the active layer, the knife coating method is particularly preferable because the active layer can be easily thickened.

The coating liquid for forming the active layer by the coating method includes, for example, a p-type semiconductor material (electron-donating compound), an n-type semiconductor material (electron-accepting compound), and a solvent.

Examples and preferable examples of the p-type semiconductor material and the n-type semiconductor material (electron-accepting compound) that can be contained in the coating liquid are the same as the examples and preferable examples described as the materials that can be contained in the active layer.

Examples of the solvent that can be contained in the coating liquid for forming the active layer include an aromatic hydrocarbon solvent (for example, toluene, xylene, trimethylbenzene, butylbenzene, methylnaphthalene, tetraline, indan, chlorobenzene, and dichlorobenzene), a ketone solvent (for example, acetone, methyl ethyl ketone, cyclohexanone, acetophenone, and propiophenone); an ester solvent (for example, ethyl acetate, butyl acetate, phenyl acetate, ethyl cellsolve acetate, methyl benzoate, butyl benzoate, and benzyl benzoate); and mixed solvents of these. As the solvents that can be contained in the coating liquid for forming the active layer, (1) an aromatic hydrocarbon solvent as the main solvent (hereinafter, also referred to as the first solvent), and (2) a solvent containing one or more solvents selected from the group consisting of a ketone solvent and an ester solvent, as the other additive solvent (referred to as the second solvent), are preferable.

Suitable combinations of the first solvent and the second solvent include, for example, a combination of o-xylene and acetophenone.

The coating liquid used in the coating method for forming the active layer may be a dispersion liquid such as an emulsion (emulsion liquid) or a suspension (suspension).

After applying the coating liquid to the coating target to form a coating film, the solvent is usually removed from the coating film. The example of the method for removing the solvent is the same as the example of the method for removing the solvent described above in the step of forming the electron transport layer.

The thickness of the active layer can be adjusted, for example, by appropriately adjusting the solid content concentration in the coating liquid, the conditions of the coating method, and the like. More specifically, in a case where the active layer is formed by coating using the knife coating method, the thickness of the active layer can be adjusted by adjusting the gap between the coated surface and the knife.

[Step (VI)]

In this step, a hole transport layer containing nanoparticles of metal oxide is formed.

Since the hole transport layer can be easily formed at low cost, it is preferable to form the hole transport layer by a coating method using a coating liquid.

Examples of the coating method for forming the hole transport layer are the same as the above-described examples of the coating method for forming the electron transport layer.

As a coating method for forming the hole transport layer, a spin coating method, a slit coating method, a flexographic printing method, an ink jet printing method, or a dispenser printing method is preferable.

The coating liquid for forming the hole transport layer by the coating method usually contains nanoparticles of metal oxide and a solvent. Examples and preferable examples of the nanoparticles of the metal oxide that can be contained in the coating liquid are the same as examples and preferable examples of the nanoparticles of the metal oxide that can be contained in the hole transport layer. Here, the solvent includes a dispersion medium.

Examples of the solvent that can be contained in the coating liquid for forming the hole transport layer are the same as the above-described examples of the coating liquid for forming the electron transport layer.

The coating liquid for forming the hole transport layer is usually a dispersion liquid such as an emulsion or suspension.

After applying the coating liquid to the coating target to form a coating film, the solvent is usually removed from the coating film. The example of the method for removing the solvent is the same as the example of the method for removing the solvent described above in the step of forming the electron transport layer.

[Step (V)]

In this step, a second electrode is formed. The second electrode is usually formed on the hole transport layer.

A method for forming a second electrode is not particularly limited. The second electrode can be formed, for example, by laminating the above-exemplified material on a layer (for example, a hole transport layer) on which a second electrode should be formed by a vacuum deposition method, a sputtering method, an ion plating method, a plating method, a coating method, or the like.

In the method for producing a light detecting element, for example, the step order may be changed according to the embodiment of the light detecting element to be produced. Further, it is not necessary to include a part of the steps (for example, a step of forming an electron transport layer). Further, any step may be included in addition to the above steps.

For example, the producing method according to the light detecting element of another embodiment may include a step of preparing a support substrate provided with a second electrode, a step of forming a hole transport layer containing nanoparticles of metal oxide, a step of forming an active layer, a step of forming an electron transport layer, and a step of forming a first electrode in this order.

[4. Application Example of Light Detecting Element]

The light detecting element according to the embodiment of the present invention described above is suitably applied to a detector provided in various electronic devices such as a workstation, a personal computer, a personal digital assistant, an access control system, a digital camera, and a medical device.

The light detecting element of the present invention can be suitably applied to, for example, an image detector (image sensor) for a solid-state imaging device such as an X-ray imaging device and a CMOS image sensor; a detector that detects a predetermined feature of a part of a living body such as a fingerprint detector, a face detector, a vein detector, and an iris detector; and a detector of an optical biosensor such as a pulse oximeter, which are included in the above-exemplified electronic device.

[5. Fingerprint Authenticating Device]

As described above, the light detecting element of the present invention can be applied to a fingerprint detector included in a fingerprint authenticating device. Therefore, a fingerprint authenticating device including the light detecting element of the present invention can be provided.

Hereinafter, among the detectors to which the light detecting element according to the embodiment of the present invention can be suitably applied, configuration examples of the image detector for a solid-state imaging device and a biometric information authenticating device (fingerprint detector for fingerprint authenticating device) will be described with reference to the drawings.

<Image Detector>

FIG. 2 is a diagram schematically illustrating a configuration example of an image detector for a solid-state imaging device.

An image detector 1 is provided with a CMOS transistor substrate 20, an interlayer insulating film 30 provided so as to cover the CMOS transistor substrate 20, and a photoelectric conversion element (light detecting element) 10 according to the embodiment of the present invention, provided on the interlayer insulating film 30, an interlayer wiring portion 32 that is provided so as to penetrate the interlayer insulating film 30 and electrically connects the CMOS transistor substrate 20 and the light detecting element (photoelectric conversion element) 10 to each other, a sealing layer 40 provided so as to cover the light detecting element 10, and a color filter 50 provided on the sealing layer.

The CMOS transistor substrate 20 includes any suitable configuration known in the related art in an aspect corresponding to the design.

The CMOS transistor substrate 20 includes a transistor, capacitors, and the like formed within the thickness of the substrate, and includes functional elements such as a CMOS transistor circuit (MOS transistor circuit) for realizing various functions.

Examples of the functional element include a floating diffusion, a reset transistor, an output transistor, and a selection transistor.

A signal readout circuit and the like are built in the CMOS transistor substrate 20 by such functional elements and wiring.

The interlayer insulating film 30 can be formed of any suitable insulating material known in the related art such as silicon oxide or an insulating resin. The interlayer wiring portion 32 can be formed of, for example, any suitable conductive material (wiring material) known in the related art such as copper and tungsten. The interlayer wiring portion 32 may be, for example, an in-hole wiring formed at the same time as the formation of the wiring layer, or an embedded plug formed separately from the wiring layer.

The sealing layer 40 can be formed of any suitable materials known in the related art under the conditions that the penetration of harmful substances such as oxygen and water that may functionally deteriorate the light detecting element 10 can be prevented or suppressed. The sealing layer 40 may be formed of the sealing substrate 17 described above.

As the color filter 50, for example, a primary color filter which is made of any suitable material known in the related art and which corresponds to the design of the image detector 1 can be used. Further, as the color filter 50, a complementary color filter capable of reducing the thickness as compared with the primary color filter can also be used. Examples of the complementary color filters include color filters that combine three types of (yellow, cyan, and magenta), three types of (yellow, cyan, and transparent), three types of (yellow, transparent, and magenta), and three types of (transparent, cyan, and magenta) can be used. Assuming that these are subject to the ability to generate color image data, any suitable arrangement can be made corresponding to the design of the light detecting element 10 and the CMOS transistor substrate 20.

The light received by the light detecting element 10 via the color filter 50 is converted into an electric signal according to the amount of light received by the light detecting element 10, and is output as a light receiving signal, that is, an electric signal corresponding to an image pickup target, outside the light detecting element 10.

Next, the received light signal output from the light detecting element 10 is input to the CMOS transistor substrate 20 via the interlayer wiring portion 32, and is read out by the signal readout circuit built in the CMOS transistor substrate 20, and signal processing is performed by a further optional suitable known functional unit in the related art (not shown) so as to generate the image information based on the image target.

<Fingerprint Detector>

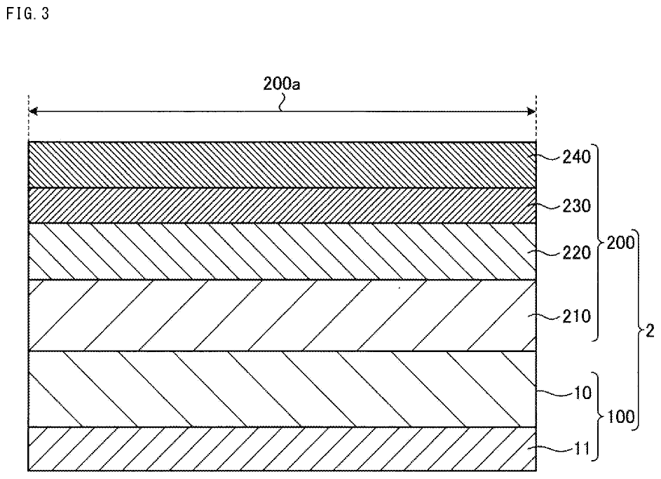

FIG. 3 is a diagram schematically illustrating a configuration example of a fingerprint detector integrated with the display device.

The display device 2 of a personal digital assistant is provided with a fingerprint detector 100 including the light detecting element 10 according to the embodiment of the present invention as a main component and a display panel unit 200 that is provided on the fingerprint detector 100, and displays a predetermined image.

In this configuration example, the fingerprint detector 100 is provided in a region that substantially coincides with a display region 200a of the display panel unit 200. In other words, the display panel unit 200 is integrally laminated above the fingerprint detector 100.

In a case where the fingerprint detection is performed only in a part region of the display region 200a, the fingerprint detector 100 may be provided corresponding to only the part region of the display region 200a. For the fingerprint detector 100, the configuration of the image detector as described above can also be adopted.

The fingerprint detector 100 includes the light detecting element 10 according to the embodiment of the present invention as a functional unit that performs an essential function. The fingerprint detector 100 can be provided with any suitable member known in the related art, such as a protection film (not shown), a support substrate, a sealing substrate, a sealing member, a barrier film, a bandpass filter, and an infrared cut film in an aspect corresponding to the design of obtaining desired characteristics.

The light detecting element 10 may be included in any aspect within the display region 200a. For example, a plurality of light detecting elements 10 may be arranged in a matrix.

As described above, the light detecting element 10 is provided on the support substrate 11 or the sealing substrate, and the support substrate 11 is provided with electrodes (positive electrode or negative electrode) in a matrix, for example.

The light received by the light detecting element 10 is converted into an electric signal according to the amount of light received by the light detecting element 10, and is output as a light receiving signal, that is, an electric signal corresponding to a captured fingerprint, outside the light detecting element 10.

In this configuration example, the display panel unit 200 is configured as an organic electroluminescence display panel (organic EL display panel) including a touch sensor panel. The display panel unit 200 may be configured of, for example, instead of the organic EL display panel, a display panel having an any suitable configuration known in the related art, such as a liquid crystal display panel including a light source such as a backlight.

The display panel unit 200 is provided on the fingerprint detector 100 as described above. The display panel unit 200 includes an organic electroluminescence element (organic EL element) 220 as a functional unit that performs an essential function. The display panel unit 200 may be further provided with any suitable substrate (support substrate 210 or sealing substrate 240) known in the related art, such as a glass substrate, a sealing member, a barrier film, a polarizing plate such as a circular polarizing plate, and any suitable member known in the related art, such as a touch sensor panel 230 in an aspect corresponding to the desired characteristics.

In the configuration example described above, the organic EL element 220 is used as a light source for pixels in the display region 200a, and is also used as a light source for capturing a fingerprint in the fingerprint detector 100.

Here, an operation of the fingerprint detector 100 will be briefly described.

When performing fingerprint authentication, the fingerprint detector 100 detects a fingerprint using the light emitted from the organic EL element 220 of the display panel unit 200. Specifically, the light emitted from the organic EL element 220 passes through the component existing between the organic EL element 220 and the light detecting element 10 of the fingerprint detector 100, and is reflected by skin (finger surface) of the fingertips of the fingers placed so as to be in contact with the surface of the display panel unit 200 within the display region 200a. At least a part of the light reflected by the finger surface passes through the components existing between them and is received by the light detecting element 10, and is converted into an electric signal corresponding to the amount of light received by the light detecting element 10. Then, image information about the fingerprint on the finger surface is formed of the converted electric signals.

The personal digital assistant provided with the display device 2 performs fingerprint authentication by comparing the obtained image information with the fingerprint data for fingerprint authentication recorded in advance by any suitable step known in the related art.

### EXAMPLES

Hereinafter, examples will be described in order to explain the present invention in more detail. The present invention is not limited to the examples described below.

In this example and the comparative example, the polymer compound represented by Formula P-1 as described above was used as the p-type semiconductor material (electron-donating compound), and C60PCBM was used as the n-type semiconductor material (electron-accepting compound). The polymer compound represented by Formula P-1 can be synthesized, for example, by referring to the method described in WO 2013/051676.

### Example 1

### Producing of Light Detecting Element (Photoelectric Conversion Element) and Evaluation Thereof

(Producing of Light Detecting Element (Photoelectric Conversion Element))

(Step of Preparing First Electrode)

A glass substrate having an ITO thin film (negative electrode) formed as a first electrode with a thickness of 150 nm was prepared by a sputtering method, and a surface of the glass substrate was subjected to an ozone UV treatment.

(Step of Forming Electron Transport Layer)

Next, a coating solution obtained by diluting polyethyleneimine ethoxylate (PEIE) (available from Aldrich, trade name: polyethyleneimine, 80% ethoxylated solution, weight average molecular weight 110000) 1/500 times with 2-methoxyethanol was applied onto an ITO thin film of a glass ITO substrate treated with ozone UV by a spin coating method.

The glass substrate coated with the coating liquid was heated at 120° C. for 10 minutes using a hot plate to form the electron transport layer 1 on the ITO thin film, which is the first electrode.

(Step of Forming Active Layer)

Next, the polymer compound represented by Formula P-1 and C60PCBM (available from Frontier Carbon Co., Ltd., trade name: E100) as an n-type semiconductor were mixed at a weight ratio of 1:2, and the mixture was added to a mixed solvent of o-xylene as the first solvent and acetophenone as the second solvent (o-xylene:acetophenone=95:5 (weight ratio)), and stirred at 80° C. for 10 hours to prepare a coating liquid for forming an active layer.

The prepared coating liquid for forming an active layer was applied onto the electron transport layer of a glass substrate by the knife coating method to obtain a coating film. The obtained coating film was dried for 5 minutes using a hot plate heated to 100° C. to form an active layer. The thickness of the formed active layer was 600 nm.

The thickness of the active layer was measured with a stylus type film thickness meter (“Surfcorder ET-200” available from Kosaka Laboratory Ltd.).

(Step of Forming Hole Transport Layer)

Next, a nickel oxide (NiO) dispersion liquid (available from Avantama, trade name: Avantama P21, average particle size: 7 nm) was applied onto the active layer by a spin coating method to obtain a coating film. The obtained coating film was dried for 5 minutes using a hot plate heated to 70° C. to form a hole transport layer 1 . The thickness of the formed hole transport layer 1 was 30 nm.

(Step of Forming Second Electrode)

Further, using a vacuum vapor deposition apparatus, a silver (Ag) layer was formed on the hole transport layer 1 as a second electrode (positive electrode) with a thickness of about 80 nm to produce a light detecting element (photoelectric conversion element).

(Sealing Step)

Next, a UV curable sealant was applied on a glass substrate around the produced light detecting element (photoelectric conversion element), the glass substrate which is the sealing substrate is bonded, and then UV light is emitted to seal the light detecting element. The planar shape of the obtained light detecting element when viewed from the thickness direction was a square of 2 mm×2 mm.

(Evaluation of Characteristics of Light Detecting Element)

The characteristics of the produced light detecting element were evaluated. The applied voltage was set to −10 V, and the dark current at this applied voltage was measured using a semiconductor parameter analyzer (Agilent Technology B1500A, produced by Agilent Technologies Japan, Ltd.). The results of the dark current measurement are indicated in Table 1.

### Examples 2 to 4, Comparative Examples 1 and 2

A light detecting element was produced and the dark current was measured in the same manner as in Example 1 except that the following items were changed.

In the step of forming the active layer, the film thickness of the active layer was adjusted to 100 nm (Comparative Example 1), 300 nm (Comparative Example 2), 750 nm (Comparative Example 2), 1400 nm (Example 3), or 2000 nm (Example 4) by adjusting the coating speed and the gap between the knife of the knife coater and the coated surface.

The results are indicated in Table 1.

### Examples 5 to 7, Comparative Examples 3 and 4

A light detecting element was produced and the dark current was measured in the same manner as in Example 1 except that the following items were changed.

In the step of forming the active layer, the thickness of the active layer was adjusted to 100 nm (Comparative Example 3), 300 nm (Comparative Example 4), 600 nm (Example 5), 750 nm (Example 6), or 1000 nm (Example 7) by adjusting the coating speed and the gap between the knife of the knife coater and the coated surface.

In the step of forming the hole transport layer, a hole transport layer 2 having a thickness of 30 nm was formed by using a tungsten oxide (WO₃) dispersion liquid (available from Avantama, trade name Avantama P10, average particle size: 10 to 20 nm) instead of the nickel oxide dispersion liquid.

The results are indicated in Table 1.

### Examples 8 and 9, Comparative Examples 5 and 6

A light detecting element was produced and the dark current was measured in the same manner as in Example 1 except that the following items were changed.

In the step of forming the active layer, the film thickness of the active layer was adjusted to 100 nm (Comparative Example 5), 300 nm (Comparative Example 6), 600 nm (Comparative 8), or 1000 nm (Example 9) by adjusting the coating speed and the gap between the knife of the knife coater and the coated surface.

In the step of forming the hole transport layer, a hole transport layer 3 having a thickness of 30 nm was formed by using a molybdenum oxide (MoO₃) dispersion liquid (available from Avantama, trade name MoO₃ in Ethanol, average particle size: about 10 nm) instead of the nickel oxide dispersion liquid.

The results are indicated in Table 1.

### Comparative examples 7 to 10

A light detecting element was produced and the dark current was measured in the same manner as in Example 1 except that the following items were changed.

In the step of forming the active layer, the film thickness of the active layer was adjusted to 100 nm (Comparative Example 7), 600 nm (Comparative Example 8), 750 nm (Comparative Example 9), or 1000 nm (Comparative Example 10) by adjusting the coating speed and the gap between the knife of the knife coater and the coated surface.

In the step of forming the hole transport layer, a hole transport layer 4 having a thickness of 30 nm was formed by using an ink containing a polythiophene derivative (available from Aldrich, trade name: Plexcore OC AQ1100) instead of the nickel oxide dispersion liquid.

The results are indicated in Table 1.

### Comparative Examples 11 to 14

A light detecting element was produced and the dark current was measured in the same manner as in Example 1 except that the following items were changed.

In the step of forming the active layer, the film thickness of the active layer was adjusted to 300 nm (Comparative Example 11), 600 nm (Comparative Example 12), 750 nm (Comparative Example 13), or 1000 nm (Comparative Example 14) by adjusting the coating speed and the gap between the knife of the knife coater and the coated surface.

In the step of forming the hole transport layer, a hole transport layer 5 having a thickness of 30 nm was formed by using PEDOT: PSS (available from Heraeus, trade name: HTL-Solar) instead of the nickel oxide dispersion liquid.

The results are indicated in Table 1.

### Comparative examples 15 to 17

A light detecting element was produced and the dark current was measured in the same manner as in Example 1 except that the following items were changed.

The thickness of the active layer was set to 600 nm (Comparative Example 15), 750 nm (Comparative Example 16), or 1000 nm (Comparative Example 17).

In the step of forming the hole transport layer, a hole transport layer 6 having a thickness of 30 nm was formed by using PEDOT: PSS (available from Heraeus, trade name: FHC-Solar) instead of the nickel oxide dispersion liquid.

The results are indicated in Table 1.

### Comparative Examples 18 to 21

A light detecting element was produced and the dark current was measured in the same manner as in Example 1 except that the following items were changed.

In the step of forming the active layer, the film thickness of the active layer was adjusted to 100 nm (Comparative Example 18), 300 nm (Comparative Example 19), 600 nm (Comparative Example 20), or 1000 nm (Comparative Example 21) by adjusting the coating speed and the gap between the knife of the knife coater and the coated surface.

In the step of forming the hole transport layer, a hole transport layer 7 having a thickness of 30 nm was formed by vacuum-depositing molybdenum oxide on the active layer.

The results are indicated in Table 1.

### Comparative Examples 22 to 26

A light detecting element was produced and the dark current was measured in the same manner as in Example 1 except that the following items were changed.

The film thickness of the active layer was set to 100 nm (Comparative Example 22), 300 nm (Comparative Example 23), 600 nm (Comparative Example 24), 750 (Comparative Example 25), or 1000 nm (Comparative Example 26).

The first electrode was formed on the active layer without performing the step of forming the hole transport layer.

The results are indicated in Table 1.

In the table below, “dark current” indicates a dark current (A/cm²) when a reverse bias voltage of 10 V is applied.

TABLE 1

| | Hole | Thickness of | |
|---|---|---|---|
| | transport | active layer | Dark current |
| | layer | (nm) | (A/cm²) |
| Comparative Example 1 | Hole | 100 | 4 × 10⁻⁶ |
| Comparative Example 2 | transport | 300 | 9 × 10⁻⁷ |
| Example 1 | layer 1 | 600 | 3 × 10⁻⁸ |
| Example 2 | | 750 | 2 × 10⁻⁸ |
| Example 3 | | 1400 | 3 × 10⁻⁸ |
| Example 4 | | 2000 | 2 × 10⁻⁸ |
| Comparative Example 3 | Hole | 100 | 6 × 10⁻⁵ |
| Comparative Example 4 | transport | 300 | 8 × 10⁻⁶ |
| Example 5 | layer 2 | 600 | 7 × 10⁻⁸ |
| Example 6 | | 750 | 7 × 10⁻⁸ |
| Example 7 | | 1000 | 8 × 10⁻⁸ |
| Comparative Example 5 | Hole | 100 | 3 × 10⁻⁴ |
| Comparative Example 6 | transport | 300 | 4 × 10⁻³ |
| Example 8 | layer 3 | 600 | 7 × 10⁻⁸ |
| Example 9 | | 1000 | 2 × 10⁻⁸ |
| Comparative Example 7 | Hole | 100 | 4 × 10⁻⁵ |
| Comparative Example 8 | transport | 600 | 2 × 10⁻⁶ |
| Comparative Example 9 | layer 4 | 750 | 2 × 10⁻⁶ |
| Comparative Example 10 | | 1000 | 1 × 10⁻⁶ |
| Comparative Example 11 | Hole | 300 | 7 × 10⁻⁵ |
| Comparative Example 12 | transport | 600 | 6 × 10⁻⁵ |
| Comparative Example 13 | layer 5 | 750 | 3 × 10⁻⁵ |
| Comparative Example 14 | | 1000 | 5 × 10⁻⁶ |
| Comparative Example 15 | Hole | 600 | 2 × 10⁻² |
| Comparative Example 16 | transport | 750 | 9 × 10⁻³ |
| Comparative Example 17 | layer 6 | 1000 | 4 × 10⁻³ |
| Comparative Example 18 | Hole | 100 | 5 × 10⁻⁵ |
| Comparative Example 19 | transport | 300 | 1 × 10⁻⁵ |
| Comparative Example 20 | layer 7 | 600 | 7 × 10⁻⁶ |
| Comparative Example 21 | | 1000 | 3 × 10⁻⁷ |
| Comparative Example 22 | No hole | 100 | 4 × 10⁻² |
| Comparative Example 23 | transport | 300 | 3 × 10⁻⁴ |
| Comparative Example 24 | layer | 600 | 3 × 10⁻⁴ |
| Comparative Example 25 | | 750 | 6 × 10⁻⁵ |
| Comparative Example 26 | | 1000 | 4 × 10⁻⁶ |

According to the above results, it is understood that in the light detecting element of Examples 1 to 9 in which the hole transport layer contains nanoparticles of metal oxide and the thickness of the active layer is 600 nm or more, the dark current is significantly reduced as compared with the light detecting element of Comparative Example 1 or 2 in which a thickness of the active layer is smaller than 600 nm, the light detecting element of Comparative Examples 7 to 21 not containing nanoparticles of metal oxide, and the light detecting element of Comparative Examples 22 to 26 not containing the hole transport layer.

### DESCRIPTION OF REFERENCE SIGNS

1 Image detector
2 Display device
10 Light detecting element
11, 210 Support substrate
12 First electrode (negative electrode)
13 Electron transport layer
14 Active layer
15 Hole transport layer
16 Second electrode (positive electrode)
17 Sealing substrate
20 CMOS transistor substrate
30 Interlayer insulating film
32 Interlayer wiring portion
40 Sealing layer
50 Color filter
100 Fingerprint detector
200 Display panel unit
200a Display region
220 Organic EL element
230 Touch sensor panel
240 Sealing substrate

1. A light detecting element comprising:
a first electrode and a second electrode;
an active layer provided between the first electrode and the second electrode; and
a hole transport layer provided between the second electrode and the active layer,
wherein the active layer contains an organic compound, and the thickness of the active layer is 600 nm or more, and
the hole transport layer contains nanoparticles of metal oxide.

2. The light detecting element according to claim 1, wherein the metal oxide contains at least one selected from the group consisting of a molybdenum atom, a tungsten atom, and a nickel atom.

3. The light detecting element according to claim 2, wherein the metal oxide is at least one selected from the group consisting of molybdenum oxide, tungsten oxide, and nickel oxide.

4. The light detecting element according to claim 1, wherein the active layer contains a conjugated polymer compound.

5. The light detecting element according to claim 1, wherein the active layer contains a fullerene derivative.

6. A fingerprint authenticating device comprising: the light detecting element according to claim 1.

7. The light detecting element according to claim 2, wherein the active layer contains a conjugated polymer compound.

8. The light detecting element according to claim 3, wherein the active layer contains a conjugated polymer compound.

9. The light detecting element according to claim 2, wherein the active layer contains a fullerene derivative.

10. The light detecting element according to claim 3, wherein the active layer contains a fullerene derivative.

11. The light detecting element according to claim 4, wherein the active layer contains a fullerene derivative.

12. A fingerprint authenticating device comprising: the light detecting element according to claim 2.

13. A fingerprint authenticating device comprising: the light detecting element according to claim 3.

14. A fingerprint authenticating device comprising: the light detecting element according to claim 4.

15. A fingerprint authenticating device comprising: the light detecting element according to claim 5.
